(19) United States
(12) Patent Application Publication
(10) Pub. No.: US 2024/0245693 A1
(43) Pub. Date: Jul. 25, 2024

(54) COMPOSITIONS AND METHODS OF TISSUE REGENERATION THERAPY

(71) Applicant: The Regents of the University of California, Oakland, CA (US)

(72) Inventors: Ichiro NISHIMURA, Venice, CA (US); Akishige HOKUGO, Los Angeles, CA (US); Takeru KONDO, Los Angeles, CA (US); Yoichiro SHIBUYA, Los Angeles, CA (US)

(73) Assignee: The Regents of the University of California, Los Angeles, CA (US)

(21) Appl. No.: 18/289,823

(22) PCT Filed: May 20, 2022
(86) PCT No.: PCT/US22/30357

§ 371 Date: Nov. 7, 2023

Related U.S. Application Data
(60) Provisional application No. 63/190,897, filed on May 20, 2021.

(51) Int. Cl. A61K 31/5377; A61K 31/40; A61K 31/41; A61K 31/438; A61K 31/4745; A61K 31/48; A61K 31/496; A61K 45/06; A61P 17/02

(52) U.S. Cl. CPC A61K 31/5377; A61K 31/40; A61K 31/41; A61K 31/438; A61K 31/4745; A61K 31/48; A61K 31/496; A61K 45/06; A61P 17/02

(57) ABSTRACT

Using a reversed-phase high-performance liquid chromatographic (HPLC) technique, a mixture of antimycins A was separated into eight hitherto unreported subcomponents, Ala, Alb, A2a, A2b, A3a, A3b, Ma, and A4b. Although a base-line resolution of the known four major antimycins Al, A2, A3, and A4 was readily achieved with mobile phases containing acetate buffers, the separation of the new antibiotic subcomponents was highly sensitive to variation in mobile phase conditions. The type and composition of organic modifiers, the nature of buffer salts, and the concentration of added electrolytes had profound effects on capacity factors, separation factors, and peak resolution values. Of the numerous chromatographic systems examined, a mobile phase consisting of methanol-water (70:30) and 0.005 M tetrabutylammonium phosphate at pH 3.0 yielded the most satisfactory results for the separation of the subcomponents. Reversed-phase gradient HPLC separation of the dansylated or methylated antibiotic compounds produced superior chromatographic characteristics and the presence of added electrolytes was not a critical factor for achieving separation. Differences in the chromatographic outcome between homologous and structural isomers were interpretated based on a differential solvophobic interaction rationale. Preparative reversed-phase HPLC under optimal conditions enabled isolation of pure samples of the methylated antimycin subcomponents for use in structural studies.

### FIELD OF THE INVENTION

The present disclosure relates in general to the fields of wound healing and tissue regeneration therapy. In one embodiment, the present disclosure provides methods of using agents that suppress expression of neuronal PAS domain protein 2 (Npas2) for wound healing, tissue regeneration and prevention of scar formation during wound healing.

### BACKGROUND OF THE INVENTION

Unclosed open wounds of the skin and oral cavity present a major threat for the current medical and dental treatment. The therapeutic goals of soft tissue wound management are infection control and wound closure. When Eliason and McLaughlin published a classic review in 1934 on postoperative wound complications, their focus was largely on surgical site infections. The effective use of antibiotics and aseptic procedures has significantly reduced the risk of surgical site infection today. However, the challenge to enhance wound healing and limit scarring remains problematic. Current approaches to achieve wound closure employ sutures and adhesives that have gone essentially unchanged for over a century.

The face and head are among the most frequent regions for wounding, which can occur due to accidents, assaults or battlefield injury. Facial wounds account for about 4% to 7% of all emergency department visits and emergency department treats nearly 90% of facial soft tissue injuries, with a wide variety of wound closure methods available to clinicians. While major facial injuries, such as facial cancers, burns or fractures obviously lead to numerous social consequences for patients, even minor facial injuries can exhibit significant psychosocial impact, resulting in a decreased satisfaction with life, an altered perception of body image, and higher incidences of posttraumatic stress disorder, alcoholism, jail, unemployment or marital problems.

Chronic skin wounds do not progress through an orderly and timely sequence of repair, resulting in the failure to achieve anatomic and functional dermal tissue integrity. A 1% to 2% prevalence of chronic non-healing wounds was reported in the general population in Germany. In the U.S., over 6 million people are affected with significant morbidity and mortality, leading to the high medical cost estimated at $28 billion to $96.8 billion toward the wound care of Medicare beneficiaries.

The primary lesion of periodontitis in the oral cavity presents an open space between gingiva and the tooth surface, termed periodontal pocket, which provides an abnormal environment for the oral microbiome, resulting in the growth of pathogenic bacteria. The closure of the periodontal pocket is currently achieved only by pocket reduction surgery. The National Health and Nutrition Examination Survey of the U.S. civilian non-institutionalized population reported that 46% of dentate adults, representing 64.7 million people, suffered from periodontitis. The prevalence of periodontitis was positively associated with increasing age, and with 8.9% of the people having developed severe or aggressive periodontitis. Similarly, periodontitis is the most widely experienced oral disease in companion dogs. According to the American Veterinary Dental College, periodontal disease is the most common clinical condition and the prevalence may reach over 90% in some dog breeds, presenting a large unmet veterinary patient population.

The circadian rhythm (also known as circadian clock), known as endogenous self-sustained and cell-autonomous oscillations of 24 hour rhythms in mammalian cells, is responsible for a wide range of physiological homeostasis functions, and the disruption of this rhythm is involved in chronic diseases, such as cardiovascular disease, diabetes, metabolic and sleep disorders, infertility, and impaired wound healing. Circadian clock has been reported to regulate physiological tissue regeneration in adult animals. Core circadian clock (rhythm) is rigidly maintained in the central brain by the suprachiasmatic nuclei (SCN) in the hypothalamus, which is the circadian pacemaker. Clock molecules: Clock, Npas2 and Bmal1 transcription factors induce the expression of Per and Cry genes, the protein products of which, in turn, inhibit Clock, Npas2 and Bmal1 transcriptional activity. In addition to core circadian clock (feed forward/back system in SCN), peripheral tissues such as bone, liver, skin and heart maintain their own circadian clock (e.g., clock molecule expression). Mouse calvarial bone organ culture demonstrated the bone mineral deposition in a circadian cycle. A microarray analysis of mouse calvaria revealed the presence of peripheral circadian rhythm in bone and that the daily expression of nearly 30% of all genes followed the 24-hour cycle, known as clock-controlled genes (CCG). Peripheral circadian clock is shown to play a regulatory role in cutaneous wound and bone fracture healing.

One of the circadian rhythm core regulators, neuronal PAS domain protein2 (NPAS2) is a member of the basic helix-loop-helix (bHLH)-PAS family of transcription factors and is a paralog of the circadian locomotor output cycles kaput (CLOCK). NPAS2 or CLOCK dimerizes with brain and muscle Arnt-like protein-1 (BMAL1) to regulate the gene transcription of two other circadian gene clusters; period (PER) and cryptochrome (CRY). PER and CRY then suppress the expression of NAPS2, CLOCK, and BMAL1 by a transcription/translation feedback loop system. Previous studies have revealed that Npas2 expression occurs in the mammalian forebrain and central brain but not in the SCN. However, distinct expression of Npas2 was reported in peripheral tissue, including the heart, liver, vasculature, and skin.

Prior studies on certain Npas2 upregulators for acceleration of osteointegration were described in PCT/US2019/059693, published as WO2020/096975, which is incorporated herein in its entirety. Prior studies on certain Npas2 suppressors for accelerating bone repair and wound healing were described in PCT/US2020/049529, published as WO2021/046428, which is incorporated herein in its entirety.

It is, therefore, of high importance to identify agents that can be administered to a wound of a subject, in particular an open dermal wound in the skin and oral cavity, to enhance wound healing, regenerate alveolar bone, regenerate connective tissue at the wound site and/or prevent scar formation at the wound site during wound healing.

### SUMMARY OF THE INVENTION

In one aspect, this disclosure provides a method for improving or accelerating wound healing in a subject comprising administering to a wound of the subject in need thereof an agent that suppresses expression of the clock gene neuronal PAS domain protein 2 (Npas2), wherein the agent is a serotonin receptor antagonist. For example, the agent is an antagonist of the serotonin receptor 1B or serotonin receptor 2B. In one embodiment, the agent is a selective antagonist of the serotonin receptor 1B or of the serotonin receptor 2B.

In another aspect, this disclosure provides a method for regenerating alveolar bone comprising administering to a bone loss site of a subject in need thereof an agent that suppresses expression of Npas2, wherein the agent is a serotonin receptor antagonist. For example, the agent is an antagonist of the serotonin receptor 1B or serotonin receptor 2B. In one embodiment, the agent is a selective antagonist of the serotonin receptor 1B or of the serotonin receptor 2B.

In a further aspect, this disclosure provides a method for regenerating connective tissue at a wound site in a subject in need thereof comprising administering to the wound a therapeutically effective amount of a Npas2 expression suppressor, wherein the suppressor is a serotonin receptor antagonist. For example, the suppressor is an antagonist of the serotonin receptor 1B or serotonin receptor 2B. In one embodiment, the agent is a selective antagonist of the serotonin receptor 1B or of the serotonin receptor 2B.

In another aspect, this disclosure provides a method for decreasing wound area size comprising topically administering to an open wound site of a subject an agent that suppresses expression of Npas2, wherein the agent is a serotonin receptor antagonist. For example, the agent is an antagonist of the serotonin receptor 1B or serotonin receptor 2B. In one embodiment, the agent is a selective antagonist of the serotonin receptor 1B or of the serotonin receptor 2B.

In still another aspect, this disclosure provides a method for reducing or preventing scar formation at a wound site during wound healing in a subject in need thereof, the method comprising administering to a wound of the subject in need thereof a therapeutically effective amount of a composition comprising an agent that suppresses expression of neuronal PAS domain protein 2 (Npas2), wherein the agent is a serotonin receptor antagonist. In certain embodiments, wherein the serotonin receptor antagonist is an antagonist of the serotonin receptor 1B or serotonin receptor 2B, as described herein. In one embodiment, the agent is a selective antagonist of the serotonin receptor 1B or of the serotonin receptor 2B.

Other features and advantages of the present invention will become apparent from the following detailed description, examples and figures. It should be understood, however, that the detailed description and the specific examples while indicating certain embodiments of the invention are given by way of illustration only, since various changes and modifications within the spirit and scope of the invention will become apparent to those skilled in the art from this detailed description. Whenever appropriate, any embodiment of the present invention can be combined with one or more other embodiments of the present invention, even though the embodiments are described under different aspects of the present invention.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1B show wound healing in mouse excisional skin wound splinting model, wherein a splinting ring is tightly adhered to the skin around the wound, thereby preventing local skin contraction. The wound is thus healed through a process of granulation and re-epithelialization similar to that occurring in human. The figures present a time course healing of skin splinted wound in wild type (WT) (FIG. 1A) and Npas2 KO mice (FIG. 1B). Npas2 KO mice demonstrated accelerated wound closure with dermal tissue regeneration.

FIG. 2 shows histological examination of mouse splinted wound healing at day 14, demonstrating WT mice wound healing was entirely by granulation tissue and scar tissue formation beyond the wound margin (white arrow). In contrast, Npas2 KO mice regenerated the dermal tissue including hair follicles and highly matured epithelium and dermal connective tissue.

FIG. 3 shows over-expression of serotonin (5-HT) receptor 1B, 2B (HTR1B, 2B) upregulated Npas2 expression, thus identifying HTR1B and HTR2B as therapeutic targets.

FIG. 4 shows in vitro collagen synthesis assay using human dermal fibroblasts. Without serotonin (5-HT) supplementation, the majority of selected compounds maintained fibroblastic collagen synthesis. In the presence of 10 μM 5-HT supplementation, serotonin receptor antagonist PRX-08066 showed high level of collagen synthesis.

FIG. 5 shows results of daily application of selected compounds at 5 micromolar in 10% DMSO vehicle solution. Treatment with serotonin receptor antagonists PRX-08066 and Cyproheptadine resulted in significant wound closure and skin tissue regeneration.

FIGS. 6A-6G show ligature-induced periodontitis in mice and alveolar bone regeneration in Npas2−/− mice after ligature removal. FIG. 6A shows ligature placement around maxillary 2^{nd} molar (M2) developed gingival inflammation (dotted line) over 14 days. FIG. 6B shows the expression of proinflammatory cytokines such as IL-17a increased in the ligature placed side of palatal gingiva. FIG. 6C shows alveolar bone loss monitored by microCT progressively increased. FIG. 6D shows gingival expression of Npas2 progressively increased. FIG. 6E shows the ligature was removed at day 14, mimicking scaling and root plaining (SRP). At day 28, gingival inflammation was subsided. In WT mice, there was a gingival deficiency (white arrows) noted around M2, which was less visible in Npas2−/− mice. FIG. 6F shows before the suture removal at day 14, WT and Npas2−/− mice showed equivalent alveolar bone loss induced by periodontitis. FIG. 6G shows while alveolar bone height of WT mice remained low, Npas2−/− mice demonstrated increased bone height, suggesting bone regeneration.*: p<0.05;***: p<0.001

FIG. 7 shows human periodontal ligament stem cells (PDLSC) overexpressing monoamine-related genes revealed that HTR2B is the therapeutic target to decrease Npas2 expression.

FIG. 8 shows in vitro osteogenic assay revealed that supplementation of HTR2B specific antagonist (PRX-08066) enhanced the differentiation of PDLSC.

FIGS. 9A-9G show the effect of HTR1B and HTR2B on human dermal fibroblasts. FIG. 9A shows the lentivirus vector carrying GFP demonstrated the successful lentivirus transduction to human dermal fibroblasts. FIGS. 9B and 9C show using quantitative PCR that the steady state mRNA level of HTR1B and HTR2B was significantly increased after the lentiviral vectors were transduced. FIG. 9D shows expression of Npas2 was significantly increased by HTR1B and HTR2B over-expression, supporting the potential role of serotonin. FIG. 9E shows that serotonin supplementation decreased the in vitro collagen synthesis of human dermal fibroblasts, and FIG. 9F shows that serotonin supplementation increased Npas2 expression of human dermal fibroblasts. FIG. 9G shows that the pathological collagen hypo-synthesis of human dermal fibroblasts was mediated by HTR1B and HTR2B over-expression.

### DETAILED DESCRIPTION OF THE INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Unless otherwise defined, all technical and scientific terms used herein have the same meaning as commonly understood by one of ordinary skill in the art to which this invention belongs. Further, unless otherwise required by context, singular terms shall include pluralities and plural terms shall include the singular.

### Circadian Rhythm and Wound Healing

As noted above, the circadian rhythm (also known as circadian clock), known as endogenous self-sustained and cell-autonomous oscillations of 24 hour rhythms in mammalian cells, is responsible for a wide range of physiological homeostasis functions, and the disruption of this rhythm is involved in chronic diseases, such as cardiovascular disease, diabetes, metabolic and sleep disorders, infertility, and impaired wound healing.

Circadian clock has been reported to regulate physiological tissue regeneration in adult animals. Core circadian clock (rhythm) is rigidly maintained in the central brain by the suprachiasmatic nuclei (SCN) in the hypothalamus, which is the circadian pacemaker. Clock molecules: Clock, Npas2 and Bmal1 transcription factors induce the expression of Per and Cry genes, the protein products of which, in turn, inhibit Clock, Npas2 and Bmal1 transcriptional activity. In addition to core circadian clock (feed forward/back system in SCN), peripheral tissues such as bone, liver, skin and heart maintain their own circadian clock (e.g., clock molecule expression). Mouse calvarial bone organ culture demonstrated the bone mineral deposition in a circadian cycle. A microarray analysis of mouse calvaria revealed the presence of peripheral circadian rhythm in bone and that the daily expression of nearly 30% of all genes followed the 24-hour cycle, known as clock-controlled genes (CCG). Peripheral circadian clock is shown to play a regulatory role in cutaneous wound and bone fracture healing.

One of the circadian rhythm core regulators, neuronal PAS domain protein2 (NPAS2) is a member of the basic helix-loop-helix (bHLH)-PAS family of transcription factors and is a paralog of the circadian locomotor output cycles kaput (CLOCK). NPAS2 or CLOCK dimerizes with brain and muscle Arnt-like protein-1 (BMAL1) to regulate the gene transcription of two other circadian gene clusters; period (PER) and cryptochrome (CRY). PER and CRY then suppress the expression of NAPS2, CLOCK, and BMAL1 by a transcription/translation feedback loop system. Previous studies have revealed that Npas2 expression occurs in the mammalian forebrain and central brain but not in the SCN. However, distinct expression of Npas2 was reported in peripheral tissue, including the heart, liver, vasculature, and skin.

Mouse skin fibroblasts have been reported to express Npas2, which might compensate for the lack of Clock expression. NPAS2 was identified among significantly upregulated genes in aging human skin by microarray analysis. Taken together, it is hypothesized that Npas2 in skin fibroblasts plays a key role in homeostatic maintenance, and therefore Npas2 is a key factor during skin wound healing. As described in the disclosure herein, this hypothesis was addressed using Npas2 knockout mice.

Recently, ectopic upregulation of Npas2 in liver was linked to fibrosis formation. Npas2 is an ortholog molecule of Clock and in the absence of Clock, Npas2 substitutes the peripheral clock function of fibroblasts. Therefore, fibrosis formation in peripheral tissues of Clock knockout mice may be contributed by pathological mechanism of substituting Npas2. It has been reported recently that Npas2 knockout (KO) mice exhibited much faster skin wound healing with minimal fibrosis.

### Peripheral Circadian Clock Genes and Wound Healing

The function and phenotype of connective tissues vary in skin and oral tissue. Dermal fibroblasts, oral fibroblasts and bone forming osteoblasts are among connective tissue cells maintaining the site-specific function and phenotype, contributing to the homeostasis of health. Wounding in a broad sense affects connective tissue cells by modifying their phenotypes resulting in scarring or loss of functions. As discussed herein, it was found that peripheral circadian clock plays a previously unrecognized role during wound healing.

In addition to the core circadian clock in SCN, peripheral tissues such as fibroblasts and osteoblasts have peripheral clocks that can function autonomously, as described by Matsui M S, Biological Rhythms in the Skin. Int J Mol Sci. 2016; 17(6), which is incorporated by reference herein in its entirety. A previous study reported that the database of human burn injuries showed that wounds injured during the night (the rest period) healed more slowly than wounds acquired during the day (the active period), as described by Hoyle N P, et al., Circadian actin dynamics drive rhythmic fibroblast mobilization during wound healing. Sci Transl Med. 2017; 9(415), which is incorporated by reference herein in its entirety. These results suggest a regulatory role of circadian rhythm in wound healing, though the mechanism of how the circadian rhythm contributes to skin wound healing is still unclear.

Mouse skin fibroblasts have been reported to express Npas2, which might compensate for the lack of Clock expression, as described by Landgraf D, et al., NPAS2 Compensates for Loss of CLOCK in Peripheral Circadian Oscillators. PLoS Genet. 2016; 12(2):e1005882, which is incorporated by reference herein in its entirety. Npas2 was identified among significantly upregulated genes in aging human skin by microarray analysis, as described by Glass D, et al., Gene expression changes with age in skin, adipose tissue, blood and brain. Genome Biol. 2013; 14(7):R75, which is incorporated by reference herein in its entirety. Recently, it has been observed that Npas2 plays a role in facilitating enhanced skin wound healing, as described by Sasaki H, et al., Neuronal PAS Domain 2 (Npas2)-Deficient Fibroblasts Accelerate Skin Wound Healing and Dermal Collagen Reconstruction. Anat Rec (Hoboken). 2019, which is incorporated by reference herein in its entirety.

As previously noted, Npas2−/− mice demonstrated faster skin wound closure than the other groups. In one embodiment, the present disclosure is directed to the application of certain small molecule compounds targeting circadian clock molecule for regenerative therapy of alveolar bone and dermal wounds. Therapeutic potential of small malecules modulating circadian systems has been proposed as a novel approach of “chronotherapy”. The present invention also is directed to small molecule-based chronotherapy for effective, safe and affordable dental tissue regeneration, including but not limited to alveolar bone regeneration, and wound healing, to patients in need thereof.

One of the major challenges in chronotherapy is selecting a target clock molecule. Because most, if not all, of cells possess circadian clock mechanisms, therapeutic modulation may result in a wide range of side effects. For example, KO mutations of Bmal1 or Clock generated various pathological phenotypes in peripheral bone tissues and premature aging symptoms (sarcopenia, cataracts, organ shrinkage). By contrast, Npas2 KO mutation did not result in embryonic and developmental pathology of jawbone, vertebral and appendicular bones. The level of Npas2 expression in SCN is low and has little contribution to the central circadian rhythm. Instead, increased Npas2 expression appears in peripheral tissues under disease states. The expression of Npas2 in bone tissue and MSC (mesenchymal stromal/stem cells) was significantly increased when exposed to titanium (Ti) biomaterial in vivo and in vitro, respectively, as described in Mengatto et al., PLoS One. 2011; 6(1):e15848 and Hassan et al., PLoS One. 2017; 12(8):e0183359, respectively, each of which is incorporated by reference herein in its entirety. The Npas2 expression in peripheral tissues may be induced by “ad hoc” bases stimulated by environmental cues including wounding. The weighed gene co-expression analysis demonstrated that Npas2 was not co-regulated with other circadian clock genes, as described by Hassan et al., PLoS One. 2017; 12(8):e0183359. Npas2 KO MSC maintained normal expression of other core clock genes, as described by Morinaga et al., Biomaterials. 2018; 192:62-74, which is incorporated by reference herein in its entirety.

Provided herein are therapeutic methods of using agent(s) that suppresses expression of the clock gene neuronal PAS domain protein 2 (Npas2) (also called an Npas2 expression suppressor, Npas2 suppressor) for wound healing, for example, for improving and/or accelerating repair and cure of the wound, for regenerating alveolar bone at a bone loss site, for regenerating connective tissue at a wound site, for decreasing wound area size at an open wound site, and for reducing or preventing scar formation at a wound site during wound healing, said methods comprising administering to the open wound site and/or the bone loss site of a subject an Npas2 expression suppressor. In one embodiment, the administered therapeutic agent(s) that suppress expression of Npas2 is an antagonist of the serotonin receptor. For example, the agent is an antagonist of the serotonin receptor 1B or serotonin receptor 2B.

As disclosed herein, Npas2 expression suppressor(s) regenerate connective tissue that has undergone a wound or chronic inflammation, regenerate dermal (skin) wounds and periodontal tissue wounds, and promote alveolar bone regeneration at a bone loss site.

In one embodiment, the present disclosure provides a method for improving or accelerating wound healing in a subject comprising administering to a wound of the subject in need thereof an agent that suppresses expression of neuronal PAS domain protein 2 (Npas2), wherein the agent is an antagonist of the serotonin receptor. For example, the agent is an antagonist of the serotonin receptor 1B or serotonin receptor 2B.

In one embodiment, the administering is by topical administration, transdermal administration and/or subcutaneous administration. In another embodiment, the wound is a dermal wound. In some embodiments, the dermal wound is a periodontal wound. In certain embodiments, the periodontal wound comprises gingival connective tissue degeneration or alveolar bone resorption. In one embodiment, the agent that suppresses expression of Npas2 accelerates human skin fibroblast migration in a cell migration assay.

In one embodiment, the transdermal administration is an application to the wound of deformable nanoscale vesicles encapsulating the agent. In an embodiment, the transdermal administration is application to the wound of a transdermal delivery system comprising a microneedle coated with the agent, a solid polymer matrix having the agent incorporated therein, a transdermal patch comprising a reservoir storing the agent and a semi-permeable membrane, a transdermal gel comprising the agent dissolved therein, a transdermal spray comprising the agent dissolved therein, or a metered dose transdermal spray comprising the agent dissolved therein.

In some embodiments, the agent that is a Npas2 expression suppressor is an antagonist of the serotonin receptor as described herein.

### Serotonin Receptor Antagonists

In one embodiment, the agent that suppresses expression of Npas2 is an antagonist of the serotonin receptor 1B. In another embodiment, the agent that suppresses expression of Npas2 is an antagonist of the serotonin receptor 2B. In one embodiment, the agent is a selective antagonist of the serotonin receptor 1B. In one embodiment, the agent is a selective antagonist of the serotonin receptor 2B. In one embodiment the agent is a serotonin receptor antagonist.

In some embodiments, the antagonist of the serotonin receptor 1B is AR-A000002, Isamoltane (CGP-361A), Metergoline, Methiothepin, SB-216641, SB-236057 or Yohimbine.

In an embodiment, the serotonin receptor 1B antagonist is AR-A000002 ((R)—N—[5-Methyl-8-(4-methylpiperazin-1-yl)-1,2,3,4-tetrahydro,-2-naphthyl]-4-morpholinobenzamide), which has the following chemical structure:

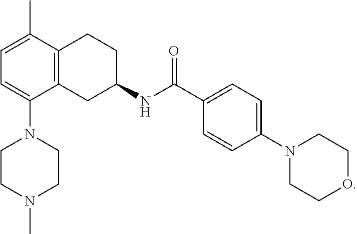 [SMILES: Cc1ccc(N2CCN(C)CC2)c2c1CC[C@@H](NC(=O)c1ccc(N3CCOCC3)cc1)C2]

In another embodiment, the serotonin receptor 1B antagonist is Isamoltane (also called CGP-361A) (2-Propanol, 1-[(1-methylethyl)amino]-3-[2-(1H-pyrrol-1-yl)phenoxy]-, hydrochloride (1:1)), which has the following chemical structure:

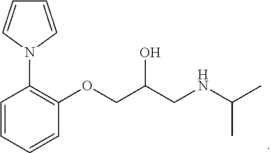 [SMILES: CC(C)NCC(O)COc1ccccc1-n1cccc1]

In an embodiment, the serotonin receptor 1B antagonist is Metergoline (Lysergamine, N-carboxy-9,10-dihydro-1-methyl-, benzyl ester), which has the following chemical structure:

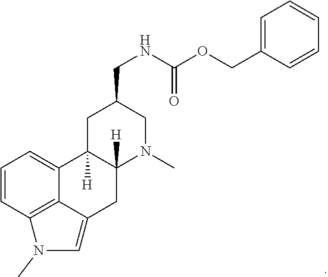

In another embodiment, the serotonin receptor 1B antagonist is Methiothepin (1-[10,11-Dihydro-8-(methylthio)dibenzo[b,f]thiepin-10-yl]-4-methylpiperazine), which has the following chemical structure:

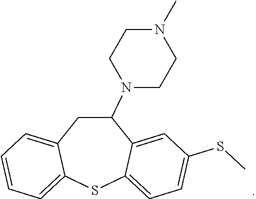 [SMILES: CSc1ccc2c(c1)C(N1CCN(C)CC1)Cc1ccccc1S2]

In one embodiment, the serotonin receptor 1B antagonist is SB-216641 (N-[3-[2-(Dimethylamino)ethoxy]-4-methoxyphenyl]-2′-methyl-4′-(5-methyl-1,2,4-oxadiazol-3-yl)[1,1′-biphenyl]-4-carboxamide), which has the following chemical structure:

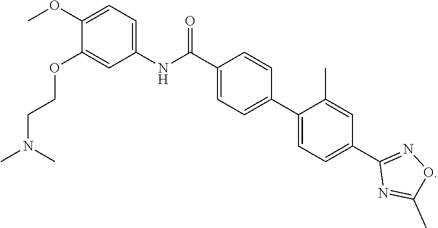 [SMILES: COc1ccc(NC(=O)c2ccc(-c3ccc(-c4noc(C)n4)cc3C)cc2)cc1OCCN(C)C]

In an embodiment, the serotonin receptor 1B antagonist is SB-236057 (1-ethyl-5-(2-methyl-4-(5-methyl-1,3,4-oxadiazolyl-2-yl)biphenyl-4-carbonyl)-2,3,6,7 tetrahydrospiro{furo[2,3-f]lindole-3,4-piperidine}hydrochloride), which is a selective inverse agonist of the serotonin receptor 1B and has the following chemical structure:

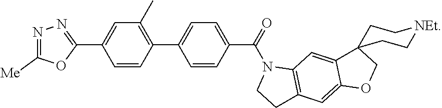

In on embodiment, the serotonin receptor 1B antagonist is SB 244289 (9 (l′-Methyl-5-[[2′-methyl-4-(5-methyl-1,2,4-oxadiazol-3-yl) biphenyl-4-yl]carbonyl]-2,3,6,7-tetrahydrospiro[furo [2,3,-f]indole-3,4′-piperidine]oxalate), which is a potent inverse agonist of the serotonin receptor 1B and has the following chemical structure:

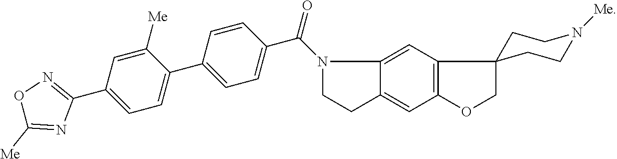 [SMILES: Cc1nc(-c2ccc(-c3ccc(C(=O)N4CCc5cc6c(cc54)C4(CCN(C)CC4)CO6)cc3)c(C)c2)no1]

In another embodiment, the serotonin receptor 1B antagonist is Yohimbine (methyl (1S,15R,18S,19R,20S)-18-hydroxy-1,3,11,12,14,15,16,17,18,19,20,21-dodecahydroyohimban-19-carboxylate), which has the following chemical structure:

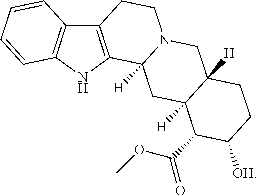 [SMILES: COC(=O)[C@@H]1[C@H]2C[C@H]3c4[nH]c5ccccc5c4CCN3C[C@@H]2CC[C@@H]1O]

In various embodiments, the antagonist of the serotonin receptor 2B is Agomelatine, Amisulpride, AM-1476, Aripiprazole, brilaroxazine hydrochloride, BF-1, BW 723C86, Cariprazine, Clozapine, Cyproheptadine, meta-Chlorophenyl-piperazine (mCPP), EGIS-7625, esamisulpride, F-16615, iferanserin, Ketanserin, LY-266097, LY-272,015, LY-287375, Metadoxine, piromelatine, Promethazine, PRX-08066, RS-127445 (MT 500). RS-127445, RQ-00310941, SB-204741, SB-206553, SB-200646A, SB-215505, SB-221284, SB 224289, SB-228357, SDZ SER-082, Tegaserod, Terguride, Ethyl 3-(4-Amino-1,3,5-triazaspiro[5.5]undeca-2,4-dien-2-ylamino)benzoate, 2-benzyl-5-hydroxy-4H-chromen-4-one, 5-hydroxy-2-(3-phenylpropyl)-4H-chromen-4-one, vabicaserin, XC-130, 4-(6-Isopopyl-1-methoxy-thioxanthen-9-yliden)-1-methyl-piperidine, 4-(6-Isopropyl-1-hydroxy-thioxanthen-9-yliden)-1-methyl-piperidine, 4-(6-Ethoxy-1-ethyl-thioxanthen-9-yliden)-1-methyl-piperidine, 4-(6-Ethoxy-1-methoxy-thioxanthen-9-yliden)-1-methyl-piperidine, 4-(6-Ethoxy-1hydoxy-thioxanthen-9-yliden)-1-methyl-piperidine, 4-(6-Methoxy-1-methoxy-thioxanthen-9-yliden)-1-methyl-piperidine, 4-(6-Dimethylamino-1-methoxy-thioxanthen-9-yliden)-1-methyl-piperidine, 4-(6-Dimethylamino-1-hydroxy-thioxanthen-9-yliden)-1-methyl-piperidine, 4-(3-Ethoxy-selenoxanthen-9-yliden)-1-methyl-piperidine, 4-(3-Ethoxy-1-methoxy-selenoxanthen-9-yliden)-1-methyl-piperidine, 4-(3-Ethoxy-1-hydroxy-selenoxanthen-9-yliden)-1-methyl-piperidine, 3-Ethoxy-9-(1-methyl-piperidine-4-ylidene)-9,10-dihydro-acridine. 6-Ethoxy-1-methoxy-9-(1-methyl-piperidine-4-ylidene)-9,10-dihydro-acridine, 6-Ethoxy-1-hydroxy-9-(1-methyl-piperidine-4-ylidene)-9, 10-dihydro-acridine, 4-(3-Ethoxy-5-ethyl-thioxanthen-9-yliden)-1-methyl-piperidine, 4-(3-Ethoxy-4-methyl-thioxanthen-9-yliden)-1-methyl-piperidine, 4-(3-Ethoxy-4-ethyl-thioxanthen-9-yliden)-1-methyl-piperidine, 4-(3-Ethoxy-thioxanthen-9-yliden)-1,3-dimethyl-piperidine, 4-(3,4-(Cyclopent-3′-oxy-1′-eno)-thioxanthen-9-yliden)-1-methyl-piperidine, 4-(3,4-(Cyclopent-4‘-oxy-1’-eno)-thioxanthen-9-yliden)-1-methyl-piperidine, 4-(3,4-(Cyclopent-5‘-oxy-1’-eno)-thioxanthen-9-yliden)-1-methyl-piperidine or a dual 5-HT2a/5-HT2b antagonist selected from the group consisting of GSK-1606260, GSK-1645888, GSK-1742942, GSK-2054757, GSK-2080717 and SB-742888.

In one embodiment, the serotonin receptor 2B antagonist is PRX-08066 (5-[[4-[(6-chlorothieno[2,3-d]pyrimidin-4-yl)amino]piperidin-1-yl]methyl]-2-fluorobenzonitrile, or a maleic acid salt thereof), a HTR2B specific antagonist, which has the following chemical structure:

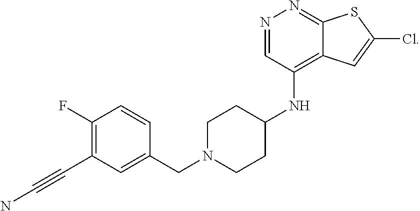 [SMILES: NC#Cc1cc(CN2CCC(Nc3cnnc4sc(Cl)cc34)CC2)ccc1F]

In one embodiment, the serotonin receptor 2B antagonist is cyproheptadine (1-methyl-4-(2-tricyclo[9.4.0.03,8]pentadeca-1(15),3,5,7,9,11,13-heptaenylidene)piperidine), sold under the name PERIACTIN, which has the following chemical structure:

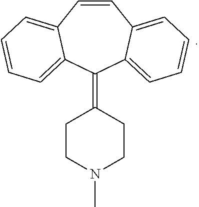 [SMILES: CN1CCC(=C2c3ccccc3C=Cc3ccccc32)CC1]

In one embodiment, the serotonin receptor 2B antagonist is ketanserin (3-[2-[4-(4-Fluorobenzoyl)-1-piperidinyl]ethyl]-2,4[1H, 3H]-quinazolinedione tartrate), sold as SUFREXAL, which has the following chemical structure:

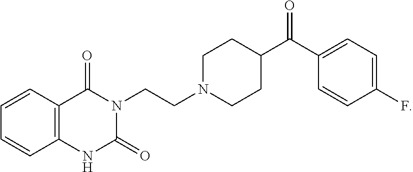 [SMILES: O=C(c1ccc(F)cc1)C1CCN(CCn2c(=O)[nH]c3ccccc3c2=O)CC1]

In one embodiment, the serotonin receptor 2B antagonist is aripiprazole (7-[4-[4-(2,3-dichlorophenyl)piperazin-1-yl]butoxy]-3,4-dihydro-1H-quinolin-2-one) which has the following chemical structure:

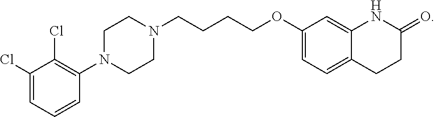 [SMILES: O=C1CCc2ccc(OCCCCN3CCN(c4cccc(Cl)c4Cl)CC3)cc2N1]

In one embodiment, the serotonin receptor 2B antagonist is sarpogrelate ((−)-4-[1-dimethylamino-3-[2-[2-(3-methoxyphenyl)ethyl]phenoxy]propan-2-yl]oxy-4-oxobutanoic acid), a mixed HTR2A and HTR2B antagonist, sold as ANPLAG, which has the following chemical structure:

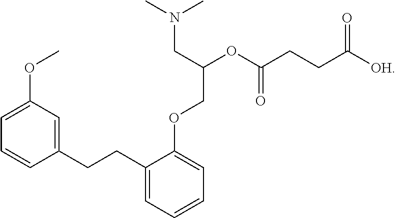 [SMILES: COc1cccc(CCc2ccccc2OCC(CN(C)C)OC(=O)CCC(=O)O)c1]

In one embodiment, the serotonin receptor 2B antagonist is RS-127445 (4-(4-fluoro-1-naphthalenyl)-6-(1-methylethyl)-2-pyrimidinamine hydrochloride), which has the following chemical structure:

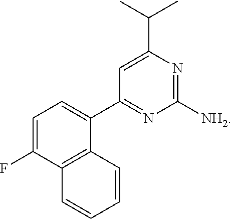 [SMILES: CC(C)c1cc(-c2ccc(F)c3ccccc23)nc(N)n1]

In one embodiment, the serotonin receptor 2B antagonist is EGIS-7625 (5-(4-benzylpiperazin-1-yl)-2-methyl-4-nitroaniline), which has the following chemical structure:

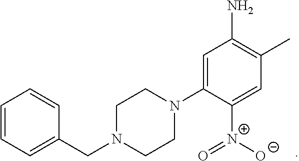 [SMILES: Cc1cc([N+](=O)[O-])c(N2CCN(Cc3ccccc3)CC2)cc1N]

In one embodiment, the serotonin receptor 2B antagonist is SB-228357 (N-(3-(3-Pyridinyl)-5-fluorophenyl)-5-methoxy-6-(trifluoromethyl)indoline-1-carboxamide), which has the following chemical structure:

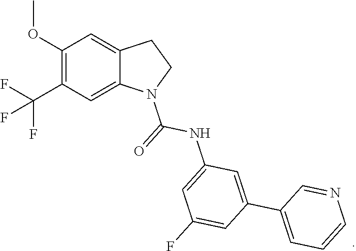 [SMILES: COc1cc2c(cc1C(F)(F)F)N(C(=O)Nc1cc(F)cc(-c3cccnc3)c1)CC2]

In one embodiment, the serotonin receptor 2B antagonist is teguride (N,N-Diethyl-N′-[(8a)-6-methylergolin-8-yl]urea), which has the following chemical structure:

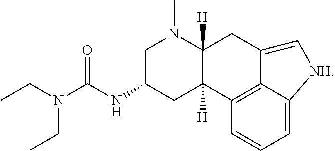 [SMILES: CCN(CC)C(=O)N[C@H]1C[C@@H]2c3cccc4[nH]cc(c34)C[C@H]2N(C)C1]

In one embodiment, the serotonin receptor 2B antagonist is SB-204741 (N-(1-methyl-1H-5-indolyl)-N′-(3-methyl-5-isothiazolyl)urea), which has the following chemical structure:

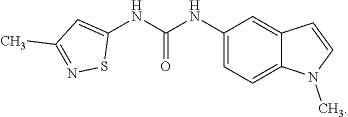 [SMILES: Cc1cc(NC(=O)Nc2ccc3c(ccn3C)c2)sn1]

In an embodiment, the serotonin receptor 2B antagonist is a semisynthetic ergot alkaloid that is a competitive alpha1-adrenergic receptor blocker and a partial alpha2-adrenergic receptor agonist. In various embodiments, the ergot alkaloid that is a serotonin (5-hydroxytryptamine or ″5-HT′) receptor antagonist, is methysergide (also called “1-methyl-D-lysergic acid butanolamide”, “UML-491”, and “methysergide maleate”, or a salt thereof), which is a serotonin 5-HT_{2c} receptor antagonist having the following chemical structure:

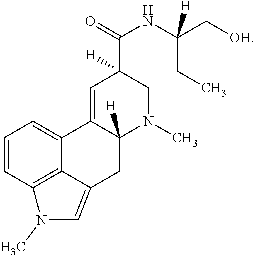

In another embodiment, the serotonin receptor 2B antagonist is amesergide (also called “N-Cyclohexyl-11-isopropyllysergamide” and “LY-237733”), which is a selective antagonist of serotonin 5-HT_{2A}, 5-HT_{2B}, and 5-HT_{2c} receptors and a potent antagonist of the α₂-adrenergic receptor, amesergide is related to methysergide and has the following chemical structure:

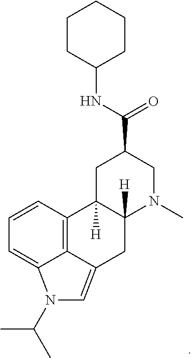

In an embodiment, the semisynthetic ergot alkaloid that is a serotonin receptor 2B antagonist is methylergometrine (also called “methylergonovine”, “methylergobasin”, “D-lysergic acid 1-butanolamide” and its salt methylergonovine maleate (Methergine®)), an active metabolite of methysergide, that is a partial agonist/antagonist of serotonergic, dopaminergic and alpha-adrenergic receptors and has the following chemical structure:

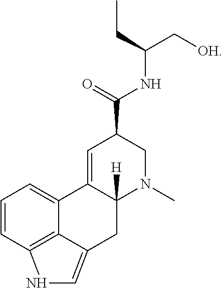

In one embodiment, the antagonist of the serotonin receptor 2B has the following chemical structure:

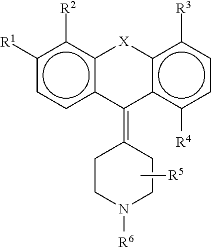

wherein R is selected from the group consisting of ethyl, propyl, isopropyl, butyl, isobutyl, pentyl, hexyl, ethoxy, propoxy, isopropoxy, butoxy, isobutoxy, phenoxy, trifluoromethyl, trifluoromethoxy, amino, dimethylamino, —CON(CH₃)₂ and —CON(C₂H₅)₂;
R² is a methyl, ethyl, propyl, isopropyl, butyl, isobutyl, pentyl, hexyl, hydroxy or hydrogen, or R¹ and R² together form a five-membered heterocycle, wherein a heteroatom in said heterocycle is an oxygen atom;
R³ is selected from the group consisting of methyl, ethyl, propyl, isopropyl, butyl isobutyl, pentyl, hexyl, hydroxy and hydrogen;
R⁴ is selected from the group consisting of hydroxy, methoxy, ethoxy, propoxy, isopropoxy, butoxy, isobutoxy, trifluoromethyl, amino, dimethylamino, diethylamino, fluorine, chlorine, bromine, methyl, ethyl, propyl, isopropyl, butyl and hydrogen;
R⁵ is methyl or hydrogen; and
R⁶ is methyl or ethyl; and X is S, N or Se, as described in WO2011009012 and U.S. Pat. No. 7,060,711, each of which is incorporated herein in its entirety.

In some embodiments, the antagonist of the serotonin receptor 2B is Agomelatine (N-[2-(7-methoxynaphthalen-1-yl)ethyl]acetamide), which has the following chemical structure:

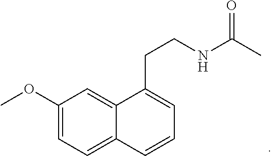 [SMILES: COc1ccc2cccc(CCNC(C)=O)c2c1]

In some embodiments, the antagonist of the serotonin receptor 2B is Amisulpride (4-amino-N-[(1-ethylpyrrolidin-2-yl)methyl]-5-ethylsulfonyl-2-methoxybenzamide), which has the following chemical structure:

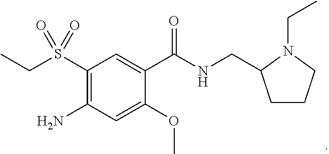 [SMILES: CCN1CCCC1CNC(=O)c1cc(S(=O)(=O)CC)c(N)cc1OC]

In an embodiment, the antagonist of the serotonin receptor 2B is Cariprazine (N′-[trans-4-[2-[4-(2,3-Dichlorophenyl)-1-piperazinyl]ethyl]cyclohexyl]-N,N-dimethylurea), which has the following chemical structure:

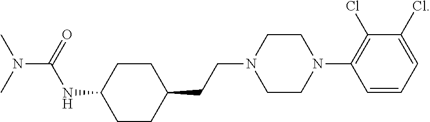 [SMILES: CN(C)C(=O)N[C@H]1CC[C@H](CCN2CCN(c3cccc(Cl)c3Cl)CC2)CC1]

In another embodiment, the antagonist of the serotonin receptor 2B is Clozapine (3-chloro-6-(4-methylpiperazin-1-yl)-11H-benzo[b][1,4]benzodiazepine; hydrochloride), which has the following chemical structure:

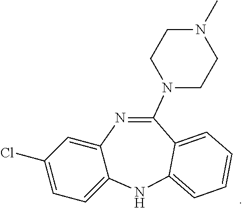 [SMILES: CN1CCN(C2=Nc3cc(Cl)ccc3Nc3ccccc32)CC1]

In an embodiment, the antagonist of the serotonin receptor 2B is meta-Chlorophenylpiperazine (mCPP), which has the following chemical structure:

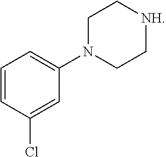 [SMILES: Clc1cccc(N2CCNCC2)c1]

In some embodiments, the antagonist of the serotonin receptor 2B is Lisuride, a dopamine agonist of the ergoline class, that is also a 5-HT2B antagonist and a dual 5-HT2A/C agonist, which has the following chemical structure:

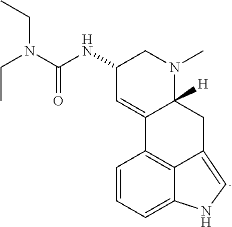

In an embodiment, the antagonist of the serotonin receptor 2B is RS-127445 (4-(4-Fluoro-1-naphthalenyl)-6-(1-methylethyl)-2-pyrimidinamine or a salt thereof, e.g., HCl), which has the following chemical structure:

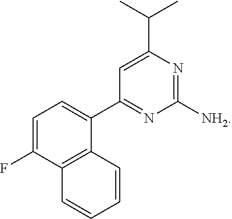 [SMILES: CC(C)c1cc(-c2ccc(F)c3ccccc23)nc(N)n1]

In some embodiments, the antagonist of the serotonin receptor 2B is Tegaserod (2-[(5-methoxy-1H-indol-3-yl)methylene]-N-pentylhydrazinecarboximidamide), which has the following chemical structure:

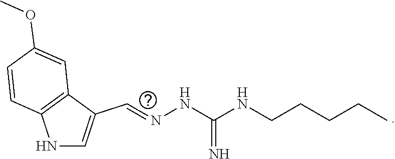 [SMILES: CCCCCNC(=N)N/N=C/c1c[nH]c2ccc(OC)cc12]

In an embodiment, the antagonist of the serotonin receptor 2B is Metadoxine (3,4-Pyridinedimethanol, 5-hydroxy-6-methyl-, compd. with 5-oxo-L-proline (1:1)), which has the following chemical structure:

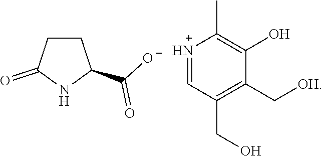

In another embodiment, the antagonist of the serotonin receptor 2B is Promethazine (N,N,α-Trimethyl-10H-phenothiazine-10-ethanamine), which has the following chemical structure:

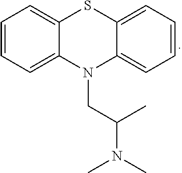 [SMILES: CC(CN1c2ccccc2Sc2ccccc21)N(C)C]

In still another embodiment, the antagonist of the serotonin receptor 2B is SDZ SER-082 (rel-(7aR,11 aS)-4,5,7a,8,9,10,11,11a-Octahydro-10-methyl-7H-indolo[1,7-bc][2,6]naphthyridine), which has the following chemical structure:

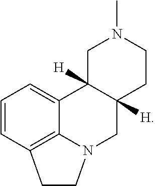

In an embodiment, the antagonist of the serotonin receptor 2B is SB-200646 N-(1-Methyl-1H-indol-5-yl)-N′-3-pyridinylurea), which has the following chemical structure:

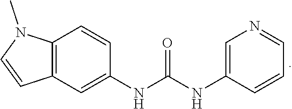 [SMILES: Cn1ccc2cc(NC(=O)Nc3cccnc3)ccc21]

In another embodiment, the antagonist of the serotonin receptor 2B is SB-204741 (N-(1-Methyl-1H-indol-5-yl)-N′-(3-methyl-5-isothiazolyl)urea), which has the following chemical structure:

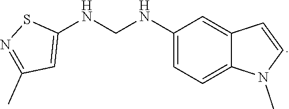 [SMILES: Cc1cc(NCNc2ccc3c(ccn3C)c2)sn1]

In an embodiment, the antagonist of the serotonin receptor 2B is SB-206553 (3,5-Dihydro-5-methyl-N-3-pyridinylbenzo[1,2-b:4,5-b′]dipyrrole-1(2H)-carboxamide or its HCl salt), which has the following chemical structure:

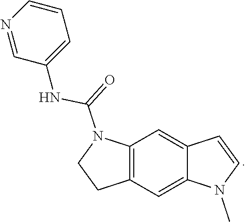 [SMILES: Cn1ccc2cc3c(cc21)CCN3C(=O)Nc1cccnc1]

In another embodiment, the antagonist of the serotonin receptor 2B is SB-215505 (6-chloro-2,3-dihydro-5-methyl-N-5-quinolinyl-1H-indole-1-carboxamide), which has the following chemical structure:

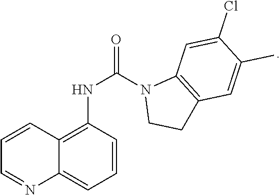 [SMILES: Cc1cc2c(cc1Cl)N(C(=O)Nc1cccc3ncccc13)CC2]

In still another embodiment, the antagonist of the serotonin receptor 2B is LY-266,097 (1-(2-Chloro-3,4-dimethoxy-benzyl)-6-methyl-2,3,4,9-tetrahydro-1H-beta-carboline), which has the following chemical structure:

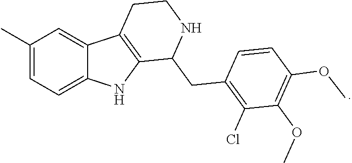

In another embodiment, the antagonist of the serotonin receptor 2B is LY-272,015 (1-[(3,4-Dimethoxyphenyl)methy]-2,3,4,9-tetrahydro-6-methyl-1H-pyrido[3,4-b]indole hydrochloride), which has the following chemical structure:

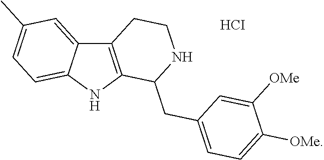

In another embodiment, the antagonist of the serotonin receptor 2B is RS-127445 ((4-(4-fluoro-1-naphthalenyl)-6-(1-methylethyl)-2-pyrimidinamine hydrochloride), which has the following chemical structure:

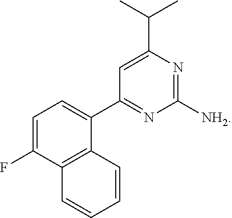 [SMILES: CC(C)c1cc(-c2ccc(F)c3ccccc23)nc(N)n1]

In still another embodiment, the antagonist of the serotonin receptor 2B is Ethyl 3-(4-Amino-1,3,5-triazaspiro[5.5]undeca-2,4-dien-2-ylamino)benzoate (as described by Zhou et al., 2016, which is incorporated herein by reference in its entirety), which has the following chemical structure:

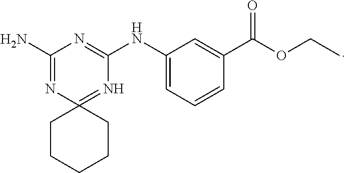

In some embodiments, the antagonist of the serotonin receptor 2B is 2-benzyl-5-hydroxy-4H-chromen-4-one, (as described by WO2015116460, which is incorporated herein by reference in its entirety), which has the following chemical structure:

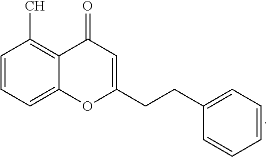 [SMILES: Cc1cccc2oc(CCc3ccccc3)cc(=O)c12]

In another embodiment, the antagonist of the serotonin receptor 2B is 5-hydroxy-2-(3-phenylpropyl)-4H-chromen-4-one, (as described by WO2015116460, which is incorporated herein by reference in its entirety), which has the following chemical structure:

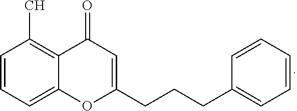 [SMILES: Cc1cccc2oc(CCCc3ccccc3)cc(=O)c12]

In some embodiments, the antagonist of the serotonin receptor 2B is RQ-00310941, (as described by Wang et al., 2021, which is incorporated herein by reference in its entirety), which has the following chemical structure:

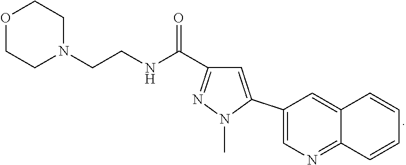 [SMILES: Cn1nc(C(=O)NCCN2CCOCC2)cc1-c1cnc2ccccc2c1]

In some embodiments, the antagonist of the serotonin receptor 2B is BF-1, (as described by Wang et al., 2021, Pharmaceuticals 14:76, which is incorporated herein by reference in its entirety), which has the following chemical structure:

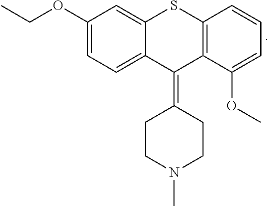 [SMILES: CCOc1ccc2c(c1)Sc1cccc(OC)c1C2=C1CCN(C)CC1]

In an embodiment, the antagonist of the serotonin receptor 2B is LY-287375 (1-[(3,4-dimethoxyphenyl)methyl]-7,8-dimethyl-2,3,4,9-tetrahydro-1H-pyrido[3,4-b]indole), which has the following chemical structure:

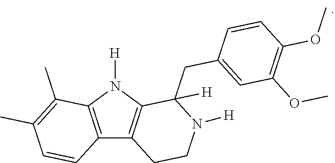

In another embodiment, the antagonist of the serotonin receptor 2B is F-16615, (as described by WO2011012868, which is incorporated herein by reference in its entirety) which has the following chemical structure:

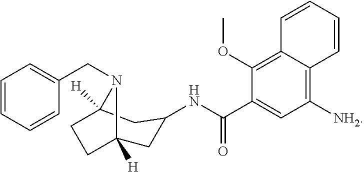 [SMILES: COc1c(C(=O)NC2C[C@H]3CC[C@@H](C2)N3Cc2ccccc2)cc(N)c2ccccc12]

In some embodiments, the antagonist of the serotonin receptor 2B has the following chemical structure:

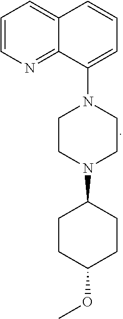 [SMILES: CO[C@H]1CC[C@H](N2CCN(c3cccc4cccnc34)CC2)CC1]

In an embodiment, the antagonist of the serotonin receptor 2B is SB-200646A (N-(1-Methyl-1H-indol-5-yl)-N′-3-pyridinylurea), which has the following chemical structure:

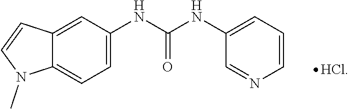 [SMILES: Cn1ccc2cc(NC(=O)Nc3cccnc3)ccc21]

In another embodiment, the antagonist of the serotonin receptor 2B is SB-221284, which has the following chemical structure:

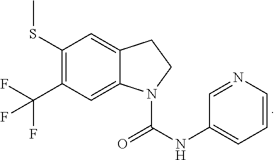 [SMILES: CSc1cc2c(cc1C(F)(F)F)N(C(=O)Nc1cccnc1)CC2]

In another embodiment, the antagonist of the serotonin receptor 2B is iferanserin, which has the following chemical structure:

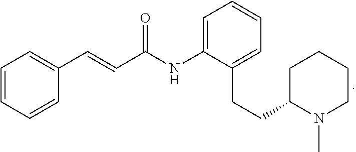 [SMILES: CN1CCCC[C@H]1CCc1ccccc1NC(=O)/C=C/c1ccccc1]

In still another embodiment, the antagonist of the serotonin receptor 2B is AM-1476, which has the following chemical structure:

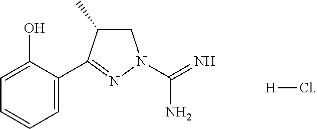 [SMILES: C[C@@H]1CN(C(=N)N)N=C1c1ccccc1O ; Cl]

In another embodiment, the antagonist of the serotonin receptor 2B is vabicaserin ((12R,16S)-7,10-diazatetracyclo[8.6.1.05,17.012,16]heptadeca-1,3,5(17)-triene), which has the following chemical structure:

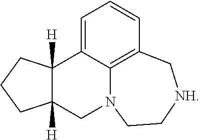 [SMILES: c1cc2c3c(c1)[C@H]1CCC[C@H]1CN3CCNC2]

In one embodiment, the antagonist of the serotonin receptor 2B is esamisulpride (S—(—)-amisulpride; S-amisulpride), which has the following chemical structure:

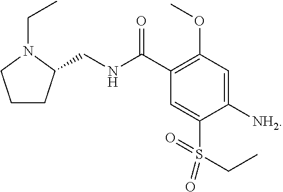 [SMILES: CCN1CCC[C@H]1CNC(=O)c1cc(S(=O)(=O)CC)c(N)cc1OC]

In another embodiment, the antagonist of the serotonin receptor 2B is brilaroxazine hydrochloride (6-(4-(4-(2,3-dichlorophenyl)piperazin-1-yl)butoxy)-2h-benzo[b][1,4]oxazin-3(4h)-one), which has the following chemical structure:

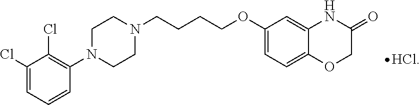 [SMILES: O=C1COc2ccc(OCCCCN3CCN(c4cccc(Cl)c4Cl)CC3)cc2N1]

In other embodiments, the antagonist is a compound disclosed in U.S. Pat. No. 10,815,235, incorporated herein by reference.

In a further embodiment, the antagonist of the serotonin receptor 2B is XC-130, also called XC130-A10H (Xoc Pharmaceuticals).

In an embodiment, the antagonist of the serotonin receptor 2B is piromelatine (N-[2-(5-Methoxy-1H-indol-3-yl)ethyl]-4-oxo-4H-pyran-2-carboxamide), which has the following chemical structure:

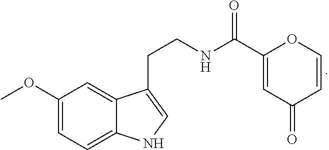 [SMILES: COc1ccc2[nH]cc(CCNC(=O)c3cc(=O)cco3)c2c1]

In some embodiments, the antagonist of the serotonin receptor 2B is SB 224289, as described by WO2011009012, and U.S. Pat. No. 7,060,711, each of which is incorporated herein by reference in its entirety.

In another embodiment, the antagonist of the serotonin receptor 2B is BW 723C86, as described by WO2011009012, which is incorporated herein by reference in its entirety.

In still another embodiment, the antagonist of the serotonin receptor 2B is selected from one of the following compounds: 4-(6-Isopropyl-1-methoxy-thioxanthen-9-yliden)-1-methyl-piperidine; 4-(6-Isopropyl-1-hydroxy-thioxanthen-9-yliden)-1-methyl-piperidine; 4-(6-Ethoxy-1-ethyl-thioxanthen-9-yliden)-1-methyl-piperidine; 4-(6-Ethoxy-1-methoxy-thioxanthen-9-yliden)-1-methyl-piperidine; 4-(6-Ethoxy-1hydroxy-thioxanthen-9-yliden)-1-methyl-piperidine; 4-(6-Methoxy-1-methoxy-thioxanthen-9-yliden)-1-methyl-piperidine; 4-(6-Dimethylamino-1-methoxy-thioxanthen-9-yliden)-1-methyl-piperidine; 4-(6-Dimethylamino-1-hydroxy-thioxanthen-9-yliden)-1-methyl-piperidine; 4-(3-Ethoxy-selenoxanthen-9-yliden)-1-methyl-piperidine; 4-(3-Ethoxy-1-methoxy-selenoxanthen-9-yliden)-1-methyl-piperidine; 4-(3-Ethoxy-1-hydroxy-selenoxanthen-9-yliden)-1-methyl-piperidine; 3-Ethoxy-9-(1-methyl-piperidine-4-ylidene)-9, 10-dihydro-acridine; 6-Ethoxy-1-hydroxy-9-(1-methyl-piperidine-4-ylidene)-9, 10-dihydro-acridine; 4-(3-Ethoxy-5-ethyl-thioxanthen-9-yliden)-1-methyl-piperidine; 4-(3-Ethoxy-4-methyl-thioxanthen-9-yliden)-1-methyl-piperidine; 4-(3-Ethoxy-4-ethyl-thioxanthen-9-yliden)-1-methyl-piperidine; 4-(3-Ethoxy-thioxanthen-9-yliden)-1,3-dimethyl-piperidine; 4-(3,4-(Cyclopent-3‘-oxy-1’-eno)-thioxanthen-9-yliden)-1-methyl-piperidine; or 4-(3,4-(Cyclopent-4‘-oxy-1’-eno)-thioxanthen-9-yliden)-1-methyl-piperidine, as described by U.S. Pat. No. 7,060,711, which is incorporated herein by reference in its entirety.

In another embodiment, this invention provides a method for regenerating alveolar bone comprising administering to a bone loss site of a subject in need thereof an agent that suppresses expression of Npas2 as described herein. In some embodiments the agent is a serotonin receptor 1B antagonist or a serotonin receptor 2B antagonist. In some embodiments, the administering is by topical administration (including gingival or periodontal administration), transdermal administration and/or subcutaneous administration. In an embodiment, the wound is a dermal wound. In another embodiment, the dermal wound is a periodontal wound. In a further embodiment, the periodontal wound comprises gingival connective tissue degeneration or alveolar bone resorption.

In one embodiment, the agent that suppresses expression of Npas2 accelerates human skin fibroblast migration in a cell migration assay. In some embodiments, the agent(s) that suppress expression of Npas2 is an antagonist of the serotonin receptor as described herein. For example, the agent is an antagonist of the serotonin receptor 1B or the serotonin receptor 2B. In one embodiment, the agent is a selective antagonist of the serotonin receptor 1B or of the serotonin receptor 2B.

In some embodiments, the transdermal (including periodontal) administration is by deformable nanoscale vesicles encapsulating the agent. In certain embodiments, the transdermal administration is application to the wound of a transdermal delivery system comprising a microneedle coated with the agent, a solid polymer matrix having the agent incorporated therein, a transdermal patch comprising a reservoir storing the agent and a semi-permeable membrane, a transdermal gel comprising the agent dissolved therein, a transdermal spray comprising the agent dissolved therein, or a metered dose transdermal spray comprising the agent dissolved therein.

In a further aspect, this invention provides a method for regenerating connective tissue at a wound site in a subject in need thereof comprising administering to the wound a therapeutically effective amount of a Npas2 expression suppressor. In some embodiments, the agent(s) that suppress expression of Npas2 is an antagonist of the serotonin receptor as described herein. For example, the agent is an antagonist of the serotonin receptor 1B or serotonin receptor 2B. In one embodiment, the agent is a selective antagonist of the serotonin receptor 1B or a selective antagonist of the serotonin receptor 2B. In some embodiments, the administering is by topical administration, periodontal administration, gingival administration, transdermal administration and/or subcutaneous administration. In one embodiment, the wound is a dermal wound. In another embodiment, the dermal wound is a periodontal wound. In certain embodiments, the periodontal wound comprises gingival connective tissue degeneration or alveolar bone resorption. In various embodiments, the agent that suppresses expression of Npas2 accelerates human skin fibroblast migration in a cell migration assay.

In some embodiments, the transdermal administration is an application to the wound of deformable nanoscale vesicles encapsulating the agent. In one embodiment, the transdermal administration is application to the wound of a transdermal delivery system comprising a microneedle coated with the agent, a solid polymer matrix having the agent incorporated therein, a transdermal patch comprising a reservoir storing the agent and a semi-permeable membrane, a transdermal gel comprising the agent dissolved therein, a transdermal spray comprising the agent dissolved therein, or a metered dose transdermal spray comprising the agent dissolved therein.

In some embodiments, the connective tissue is one or more of collagen, dermis-like collagen fibers, or bone. In one embodiment, the wound site is a site of bone loss. In another embodiment, the bone loss is a site of periodontitis-induced alveolar bone resorption. In a further embodiment, the wound site is a site of gingival connective tissue degeneration.

In another aspect, this invention provides a method for decreasing wound area size comprising topically administering to an open wound site of a subject (e.g., an incisional wound, skin graft donor site, skin grafting site, abrasion, tear, laceration, penetration, puncture, gash, cut, scrape, abrasion, scratch, thermal wound, burn, ulcer including decubitus ulcer, surgical wound or wound closure, by way of non-limiting examples), an agent that suppresses expression of Npas2. In some embodiments, the agent(s) that suppress expression of Npas2 is an antagonist of the serotonin receptor as described herein. For example, the agent is an antagonist of the serotonin receptor 1B or serotonin receptor 2B. In one embodiment, the agent is a selective antagonist of the serotonin receptor 1B or a selective antagonist of the serotonin receptor 2B. In certain embodiments, the administering is by topical administration, periodontal administration, gingival administration, transdermal administration and/or subcutaneous administration. In an embodiment, the wound is a dermal wound. In another embodiment, the dermal wound is a periodontal wound. In still another embodiment, the periodontal wound comprises gingival connective tissue degeneration or alveolar bone resorption. In an embodiment, the agent that suppresses expression of Npas2 accelerates human skin fibroblast migration in a cell migration assay. In various embodiments, the transdermal administration is an application to the wound of deformable nanoscale vesicles encapsulating the agent. In some embodiments, the transdermal administration is application to the wound of a transdermal delivery system comprising a microneedle coated with the agent, a solid polymer matrix having the agent incorporated therein, a transdermal patch comprising a reservoir storing the agent and a semi-permeable membrane, a transdermal gel comprising the agent dissolved therein, a transdermal spray comprising the agent dissolved therein, or a metered dose transdermal spray comprising the agent dissolved therein.

In another embodiment, the open wound site comprises connective tissue selected from one or more of collagen, dermis-like collagen fibers, or bone. In a further embodiment, the open wound site is a site of bone loss. In an embodiment, the bone loss is a site of periodontitis-induced alveolar bone resorption. In some embodiments, the open wound site is a site of gingival connective tissue degeneration.

In one embodiment, the agent that suppresses expression of Npas2 is formulated as a pharmaceutical composition for topical administration, periodontal administration, gingival administration, transdermal administration and/or subcutaneous administration. In some embodiments, the pharmaceutical composition comprises a therapeutically effective amount of the agent that suppresses expression of clock gene Npas2, as described herein. In certain embodiments, the pharmaceutical composition comprises a therapeutically effective amount of the agent that suppresses expression of clock gene Npas2 effective to regenerate alveolar bone at a bone loss site, to regenerate connective tissue at a wound site, and/or to decrease wound area size of a wound site, for example, an open wound site, of a subject in need thereof. In an embodiment, the pharmaceutical composition comprises at least one antagonist of the serotonin receptor that suppresses expression of clock gene Npas2. In another embodiment, pharmaceutical composition comprises a combination of serotonin receptor antagonists that suppresses expression of clock gene Npas2.

In some embodiments, the wound is an incisional wound, skin graft donor site, skin grafting site, abrasion, tear, laceration, penetration, puncture, gash, cut, scrape, abrasion, scratch, thermal wound, burn, ulcer including decubitus ulcer, surgical wound or wound closure. In some embodiments, the agent is applied regularly, such as but not limited to daily, twice a day, three times a day, or four times a day, for topical or periodontal sites; for application to surgical sites, application may be at the time of surgery, and in some embodiments, a controlled release formulation as described elsewhere herein may be provided. The frequency of application or administration to other sites and/or for other types of wounds will be prescribed by the health care professional and/or guidance found in the product label.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment”, “treating,” or “therapy” (as well as different forms thereof) of a disease-state in a mammal, particularly in a human, are used interchangeably herein and refer to (a) preventing the disease-state from occurring in a mammal, i.e., prophylaxis of the disease-state, in particular, when such mammal is predisposed to the disease-state but has not yet been diagnosed as having it; (b) inhibiting the disease-state, i.e., arresting its development, and/or curing the disease-state; and/or (c) relieving the disease-state, i.e., causing regression of the disease state. The term “treating” as used herein includes alleviating or reducing at least one adverse or negative effect or symptom of a condition, disease or disorder. Examples of disease-state include, but are not limited to, dermal wound, periodontal wound or alveolar bone loss.

In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

As used herein, the terms “administering,” “administer,” or “administration” refer to delivering one or more compounds or compositions to a subject parenterally, enterally, or topically, including any surface on the inside of the mouth. In one embodiment, the compositions are applied periodontally. In one embodiment, the compositions are applied locally. In another embodiment, the compositions are applied systemically. Administration can be accomplished to cells or tissue cultures, or to living organisms, for example humans.

Illustrative examples of parenteral administration include, but are not limited to, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. Illustrative examples of enteral administration include, but are not limited to oral, inhalation, intranasal, sublingual, and rectal administration. Illustrative examples of topical administration include, but are not limited to, transdermal and vaginal administration. In particular embodiments, an agent or composition is administered parenterally, optionally by intravenous administration or oral administration to a subject.

The terms “subject,” “individual,” and “patient” are used interchangeably herein, and refer to an animal, for example a human, to whom treatment, including prophylactic treatment and inhibition of the disease-state and secondary infection, with an agent that suppresses expression of Npas2, as described herein, and/or pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses, and non-mammals such as reptiles, amphibians, chickens, and turkeys.

According to any of the methods of the present invention and in one embodiment, a subject as described herein is human. In another embodiment, the subject is non-human. In one embodiment, the subject is a vertebrate. In another embodiment, the subject is a mammal. In another embodiment, the subject is a primate, which in one embodiment, is a non-human primate. In another embodiment, the subject is murine, which in one embodiment is a mouse, and, in another embodiment is a rat. In another embodiment, the subject is a canine, feline, bovine, equine, caprine, ovine, porcine, simian, ursine, vulpine, or lupine. In one embodiment, the subject is a chicken or fish.

In one embodiment, a composition of the present invention comprises a pharmaceutically acceptable composition. In an embodiment, the composition comprises an agent that suppresses expression of a clock gene, wherein the clock gene is neuronal PAS domain protein 2 (Npas2). In particular embodiments, the agent that suppresses expression of Npas2 is any one of the agents that suppresses expression of Npas2, as described herein. In some embodiments, the “pharmaceutically acceptable composition” and the “pharmaceutical composition” is formulated for topical administration, periodontal administration, transdermal administration and/or subcutaneous administration. In an embodiment, the “pharmaceutically acceptable composition” and the “pharmaceutical composition” comprises a pharmaceutically acceptable carrier or excipient. Topical administration includes use during surgery on any surgical site within or on the body, such as incisional wounds (e.g., internal sutured or stapled sites).

In one embodiment, the phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable” also includes those carriers (also called vehicles) approved by a regulatory agency of the Federal or a state government or listed in the U.S. Pharmacopeia or other generally recognized pharmacopeia for use in animals and, more particularly, in humans.

As used herein a “pharmaceutically acceptable carrier” or “excipient” or “vehicle”, includes solvents, dispersion media, coatings, antibacterial and antifungal agents, isotonic and absorption delaying agents, and the like that are physiologically compatible. In one embodiment, the pharmaceutically acceptable carrier is suitable for topical administration, periodontal administration, transdermal administration and/or subcutaneous administration. Topical formulations (e.g., compositions) include gels, ointments, creams, lotions, drops, powders, and the like. In some embodiments, a topical formulation comprises an active pharmaceutical ingredient (API), i.e., the compound or drug that produces the intended effects, which is an antagonist of a serotonin receptor 1B or an antagonist of the serotonin receptor 2B (or a combination thereof) in an amount of about 0.01% to about 50%, 0.05% to about 50%, about 0.1% to about 50%, about 0.5% to about 50%, about 0.5% to about 50%, about 1% to about 50%, about 5% to 50%, about 0.1% to about 5%, or about 0.01% to about 2% w/w. In some embodiments, the concentration of serotonin receptor 1B or an antagonist of the serotonin receptor 2B (or a combination thereof) in the formulation is between about 0.5 micromolar and about 10 micromolar, between about 1 micromolar and 10 micromolar, between about 1 micromolar and 5 micromolar, or between about 5 micromolar and 10 micromolar. In an embodiment, the topical formulation comprises non-active ingredients/excipients/carriers in an amount of the balance of the aforementioned ranges; in some embodiments, about 90% to 99%. The term “w/w” denotes weight/weight. In some embodiments, the topical composition comprises an antagonist of a serotonin receptor 1B or an antagonist of the serotonin receptor 2B (or a combination thereof) in an amount of about 0.001%, 0.005%,0.01%,0.02%,0.05%,0.75%, 0.1%, 0.2%,0.5%,0.75%, 1%,2%,3%,4% or 5% w/w. In various embodiments, the topical formulation comprises an antagonist of a serotonin receptor 1B or an antagonist of the serotonin receptor 2B (or a combination thereof) in an amount of about 6%, 7%, 8%, 9% or 10% w/w. In certain embodiments, the topical formulation comprises an antagonist of a serotonin receptor 1B or an antagonist of the serotonin receptor 2B (or a combination thereof) in an amount of about 11%, 12%, 13%, 14% or 15% w/w. In an embodiment, the topical formulation comprises an antagonist of a serotonin receptor 1B or an antagonist of the serotonin receptor 2B (or a combination thereof) in an amount of about 20% w/w. In another embodiment, the topical formulation comprises an antagonist of a serotonin receptor 1B or an antagonist of the serotonin receptor 2B (or a combination thereof) in an amount of about 25% w/w. In some embodiments, the topical formulation comprises an antagonist of a serotonin receptor 1B or an antagonist of the serotonin receptor 2B (or a combination thereof) in an amount of about 30% w/w. In an embodiment, the topical formulation comprises an antagonist of a serotonin receptor 1B or an antagonist of the serotonin receptor 2B (or a combination thereof) in an amount of about 35% w/w. In certain embodiments, the topical formulation comprises an antagonist of a serotonin receptor 1B or an antagonist of the serotonin receptor 2B (or a combination thereof) in an amount of about 40% w/w. In some embodiments, the topical formulation comprises an antagonist of a serotonin receptor 1B or an antagonist of the serotonin receptor 2B (or a combination thereof) in an amount of about 45% w/w. In various embodiments, the topical formulation comprises an antagonist of a serotonin receptor 1B or an antagonist of the serotonin receptor 2B (or a combination thereof) in an amount of about 50% w/w. In any of the foregoing embodiments, the antagonist is a selective antagonist of the serotonin receptor 1B or a selective antagonist of the serotonin receptor 2B.

In an embodiment, the pharmaceutically acceptable carrier is suitable for parenteral administration.

In an embodiment, the pharmaceutically acceptable carrier is suitable for periodontal administration or to a surface site within the oral cavity.

Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, i.e., the agent that suppresses expression of Npas2, use thereof in the pharmaceutical compositions described herein is contemplated. Supplementary active compounds can also be incorporated into the compositions.

Therapeutic pharmaceutical compositions typically are sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols, such as mannitol, sorbitol, or sodium chloride in the composition. Prolonged absorption of injectable compositions can be brought about by including in the composition an agent which delays absorption, for example, monostearate salts and gelatin.

Moreover, an agent that suppresses expression of Npas2, as described herein can be administered in a time release formulation, for example in a composition which includes a slow-release polymer. The agent that suppresses expression of Npas2 can be prepared with carriers that will protect it against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating an active compound, such as an agent that suppresses expression of Npas2 described herein, in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the methods of preparation include vacuum drying and freeze-drying, which yields a powder of the active ingredient plus any additional desired ingredient from a previously sterile-filtered solution thereof. In some embodiments, an agent that suppresses expression of Npas2, as described herein may be formulated with one or more additional compounds that enhance its solubility. In any of the foregoing embodiment, a sterile formulation for use within a surgical site or on a topical wound is provided.

In one embodiment, a composition of the present invention is administered in a therapeutically effective amount. In one embodiment, a “therapeutically effective amount” is intended to include an amount of an agent or compound of the present invention alone or an amount of the combination of agents or compounds claimed or an amount of an agent or compound of the present invention in combination with other active ingredients effective to act as a suppressor, inhibitor or down-regulator of expression of clock gene, neuronal PAS domain protein 2 (Npas2), effective to improve or accelerate wound healing in a subject, regenerate alveolar bone at a bone loss site of a subject, regenerate connective tissue at a wound site in a subject and/or decrease wound area size of an open wound site of a subject, to which the agent or compound is administered or has been administered. In one embodiment, a “therapeutically effective amount” of an agent that suppresses expression of clock gene Npas2 of the present invention is that amount of agent which is sufficient to provide a beneficial effect to the subject to which the composition is administered. A therapeutically effective amount of an agent administered according to the present invention, e.g., an antagonist of the serotonin receptor, wherein the antagonist of the serotonin receptor is an antagonist of the serotonin receptor 1B or an antagonist of the serotonin receptor 2B, may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects. The composition, when administered to a subject (human or animal) induces a desired pharmacological and/or physiologic effect by local and/or systemic action.

It will be appreciated that the compounds and compositions, administered according to the method of the present invention, may be administered using any amount and any route of administration effective for the treatment of conditions or diseases in which serotonin receptor antagonists, i.e., an antagonist of the serotonin receptor 1B or serotonin receptor 2B, as described herein, have a therapeutically useful role.

In certain embodiments, a pharmaceutical composition comprising an agent that suppresses expression of neuronal PAS domain protein 2 (Npas2), e.g., an antagonist of the serotonin receptor, wherein the antagonist of the serotonin receptor is an antagonist of the serotonin receptor 1B or an antagonist of the serotonin receptor 2B, may be administered at dosage levels of about 0.001 mg/kg to about 50 mg/kg, for example, from about 0.1 mg/kg to about 10 mg/kg for parenteral administration, or from about 1 mg/kg to about 50 mg/kg, or from about 10 mg/kg to about 50 mg/kg for oral administration, of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered topically, or parenterally or orally. In some embodiments, a topical formulation comprises between about 0.5 micromolar and about 10 micromolar of the compound. In some embodiments, a topical formulation comprises between about 1 micromolar and 10 micromolar, between about 1 micromolar and 5 micromolar, or between about 5 micromolar and 10 micromolar of the compound. In an embodiment, a topical pharmaceutical composition comprises a therapeutically effective amount of an agent that suppresses expression of neuronal PAS domain protein 2 (Npas2), as described herein, or a salt or solvate thereof, or a hydrate, polymorph, metabolite, tautomer or isomer thereof, and at least one pharmaceutically acceptable excipient. In some embodiments, the topical formulation comprising the antagonist of the serotonin receptor of the present invention and its physiologically tolerated derivatives such as salts, esters, N-oxides, and the like is prepared and applied as a solution, suspension, or emulsion in a physiologically acceptable diluent with or without a pharmaceutical carrier. In some embodiments, the topical pharmaceutical compositions are formulated as an ointment, cream or gel. Each possibility represents a separate embodiment of the present invention. In any of the foregoing embodiments, the agent is a selective antagonist of the serotonin receptor 1B or a selective antagonist of the serotonin receptor 2B.

The agent that suppresses expression of neuronal PAS domain protein 2 (Npas2), as described herein, is preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder, the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

For topical administration, an exemplary dosage of 50 g/m² per application for ointment base (carrier) and 40 g/m² per application for cream may be administered. At this dosage, an average individual would use (i.e., have administered) approximately 10 g of medication/week to treat 1% of the body surface (e.g., the size of a wound for accelerated healing and/or reduced scarring) though this would depend upon the size of the wound, the frequency of administration, etc. and such guidance is merely exemplary. Treatment dosages for the routes of administration described herein may be titrated using routine methods known to those of skill in the art to optimize safety and efficacy. For a topical cream composition/formulation about 1 g of cream will spread out over a 10-cm² area of skin, whereas an ointment spreads a little further. The fingertip unit (0.5 g) is a guide to the amount of a cream or ointment needed to treat an area for a certain duration; for example, 0.5 g may be administered every 2 to 12 hours, 2 to 8 hours, 2 to 6 hours, 4 to 8 hours, 4 to 6 hours, as determined by sound medical judgment. One fingertip unit covers one side of two flat hands, and one gram covers both sides of the patient's two hands. To cover (i.e., apply to) an adult's total body surface once, it takes 20-30 g of cream or ointment.

While small and moderately lipophilic molecules (molecular weight <500 Da and log P of 1-4) are likely to penetrate the skin well, some compounds/drugs that do not possess these physicochemical properties usually require a penetration enhancement strategy to penetrate the skin. In an embodiment, a topical formulation of an antagonist of the serotonin receptor, wherein the antagonist of the of the serotonin receptor is an antagonist of the serotonin receptor 1B or an antagonist of the serotonin receptor 2B, may be formulated with a chemical penetration enhancer. Penetration enhancers for transdermal drug delivery include, but are not limited to, pyrrolidones (such as 2-pyrrolidone, 2P), alcohols (such as ethanol or decanol), glycols (such as propylene glycol), esters, water, esters sulfoxides (such as dimethyl sulfoxide (DMSO)) and their derivatives, hydrocarbons, terpenes and derivatives, Azone (such as laurocapram) and its analogs, amides (including urea and its derivatives), fatty acids, surfactants (nonionic, cationic, and anionic), oleodendrimers, ionic liquids, and deep eutectic solvents. Physical penetration enhancers for transdermal drug delivery include but are not limited to microneedles and iontophoresis.

In one embodiment, the present disclosure provides a method for improving or accelerating wound healing in a subject in need thereof, comprising administering to a wound of the subject a composition comprising an agent that suppresses expression of neuronal PAS domain protein 2 (Npas2), wherein the agent is an antagonist of the serotonin receptor. In one embodiment, the agent is an antagonist of the serotonin receptor 1B. In another embodiment, the agent is an antagonist of the serotonin receptor 2B. In some embodiments, the agent is a selective antagonist of the serotonin receptor 1B or a selective antagonist of the serotonin receptor 2B. Examples of serotonin receptor antagonists include, but are not limited to, PRX-08066, cyproheptadine, or ketanserin.

In one embodiment, the wound is a dermal wound, wherein treating the dermal wound with the agent results in regeneration of skin tissue that comprises sebaceous glands and hair follicles. In another embodiment, the dermal wound comprises a periodontal wound. In one embodiment, the periodontal wound comprises gingival connective tissue degeneration or alveolar bone resorption. In one embodiment, treating the periodontal wound with the agent results in regeneration of composite periodontal tissues.

In one embodiment, the composition is administered by topical administration, periodontal administration, transdermal administration, or subcutaneous administration. In another embodiment, the pharmaceutical compositions are administered by subcutaneous implantation of a pellet. In a further embodiment, the pellet provides for controlled release of the agent that suppresses expression of neuronal PAS domain protein 2 (Npas2), wherein the agent is an antagonist of the serotonin receptor, as described herein. For example, the controlled release is from 8 to 12 hours, from 6 to 8 hours, or from 4 to 8 hours. In one embodiment, administration of the composition comprises delivering to the wound a delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

In another embodiment, the present disclosure provides a method for regenerating connective tissue at a wound site in a subject in need thereof, comprising administering to a wound of the subject a composition comprising an agent that suppresses expression of neuronal PAS domain protein 2 (Npas2), wherein the agent is an antagonist of the serotonin receptor. In one embodiment, the agent is an antagonist of the serotonin receptor 1B. In another embodiment, the agent is an antagonist of the serotonin receptor 2B. Examples of serotonin receptor antagonists include, but are not limited to, PRX-08066, cyproheptadine, or ketanserin.

In one embodiment, the wound is a dermal wound, wherein treating the dermal wound with the agent results in regeneration of skin tissue that comprises sebaceous glands and hair follicles. In another embodiment, the dermal wound comprises a periodontal wound. In one embodiment, the periodontal wound comprises gingival connective tissue degeneration or alveolar bone resorption. In one embodiment, treating the periodontal wound with the agent results in regeneration of composite periodontal tissues. In one embodiment, regeneration of composite periodontal tissues comprises one or more of regrowth of alveolar bone, regeneration of gingival ligament, and regeneration of periodontal ligament.

In one embodiment, the composition is administered by topical administration, transdermal administration, or subcutaneous administration. In one embodiment, administration of the composition comprises delivering to the wound a delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

In a further aspect, this invention provides a method for reducing or preventing scar formation at a wound site during wound healing in a subject in need thereof, the method comprising administering to a wound of the subject in need thereof a therapeutically effective amount of a composition comprising an agent that suppresses expression of neuronal PAS domain protein 2 (Npas2), wherein the agent is a serotonin receptor antagonist. In one embodiment, the agent is an antagonist of the serotonin receptor 1B. In another embodiment, the agent is an antagonist of the serotonin receptor 2B. In any of the foregoing embodiments, the agent is a selective antagonist of the serotonin receptor 1B or a selective antagonist of the serotonin receptor 2B. In an embodiment, the antagonist of the serotonin receptor 1B is Aripiprazole, AR-A000002, Isamoltane (CGP-361A), Metergoline, Methiothepin, SB-216641, SB-236057 or Yohimbine. In an embodiment, the antagonist of the serotonin receptor 2B is Agomelatine, Amisulpride, AM-1476, Aripiprazole, brilaroxazine hydrochloride, BF-1. BW 723C86, Cariprazine, Clozapine, Cyproheptadine, meta-Chlorophenyl-piperazine (mCPP), EGIS-7625, esamisulpride, F-16615, iferanserin, Ketanserin, LY-266097, LY-272,015, LY-287375, Metadoxine, piromelatine, Promethazine, PRX-08066, RS-127445 (MT 500), RS-127445, RQ-00310941, SB-204741, SB-206553, SB-200646A, SB-215505, SB-221284, SB 224289, SB-228357, SDZ SER-082, Tegaserod, Terguride. Ethyl 3-(4-Amino-1,3,5-trazaspiro[5.5]undeca-2,4-dien-2-ylamino)benzoate, 2-benzyl-5-hydroxy-4H-chromen-4-one, 5-hydroxy-2-(3-phenylpropyl)-4H-chromen-4-one, vabicaserin, XC-130, 4-(6-Isopropyl-1-methoxy-thioxanthen-9-yliden)-1-methyl-piperidine, 4-(6-Isopropyl-1-hydroxy-thioxanthen-9-yliden)-1-methyl-piperidine, 4-(6-Ethoxy-1-ethyl-thioxanthen-9-yliden)-1-methyl-piperidine, 4-(6-Ethoxy-1-methoxy-thioxanthen-9-yliden)-1-methyl-piperidine, 4-(6-Ethoxy-1hydroxy-thioxanthen-9-yliden)-1-methyl-piperidine, 4-(6-Methoxy-1-methoxy-thioxanthen-9-yliden)-1-methyl-piperidine, 4-(6-Dimethylamino-1-methoxy-thioxanthen-9-yliden)-1-methyl-piperidine, 4-(6-Dimethylamino-1-hydroxy-thioxanthen-9-yliden)-1-methyl-piperidine, 4-(3-Ethoxy-selenoxanthen-9-yliden)-1-methyl-piperidine, 4-(3-Ethoxy-1-methoxy-selenoxanthen-9-yliden)-1-methyl-piperidine, 4-(3-Ethoxy-1-hydoxy-selenoxanthen-9-yliden)-1-methyl-piperidine, 3-Ethoxy-9-(1-methyl-piperidine-4-ylidene)-9,10-dihydro-acridine, 6-Ethoxy-1-methoxy-9-(1-methyl-piperidine-4-ylidene)-9,10-dihydro-acridine, 6-Ethoxy-1-hydroxy-9-(1-methyl-piperidine-4-ylidene)-9, 10-dihydro-acridine, 4-(3-Ethoxy-5-ethyl-thioxanthen-9-yliden)-1-methyl-piperidine, 4-(3-Ethoxy-4-methyl-thioxanthen-9-yliden)-1-methyl-piperidine, 4-(3-Ethoxy-4-ethyl-thioxanthen-9-yliden)-1-methyl-piperidine, 4-(3-Ethoxy-thioxanthen-9-yliden)-1,3-dimethyl-piperidine, 4-(3,4-(Cyclopent-3‘-oxy-1’-eno)-thioxanthen-9-yliden)-1-methyl-piperidine, 4-(3,4-(Cyclopent-4′-oxy-1′-eno)-thioxanthen-9-yliden)-1-methyl-piperidine, 4-(3,4-(Cyclopent-5′-oxy-1′-eno)-thioxanthen-9-yliden)-1-methyl-piperidine or a dual 5-HT2a/5-HT2b antagonist selected from the group consisting of GSK-1606260, GSK-1645888, GSK-1742942, GSK-2054757, GSK-2080717 and SB-742888.

In one embodiment, the wound is a dermal wound, wherein treating the dermal wound by administering the agent comprises formation of a scar-reduced or scarless dermal wound. In an embodiment, treating the dermal wound by administering the agent regenerates skin tissue that comprises sebaceous glands and hair follicles. In another embodiment, the dermal wound comprises a periodontal wound. In one embodiment, the periodontal wound comprises gingival connective tissue degeneration or alveolar bone resorption. In an embodiment, treating the periodontal wound with the agent results in regeneration of composite periodontal tissues. In another embodiment, regeneration of composite periodontal tissues comprises one or more of regrowth of alveolar bone, regeneration of gingival ligament, and regeneration of periodontal ligament. In one embodiment, the composition is administered by topical administration, transdermal administration, or subcutaneous administration. In one embodiment, administration of the composition comprises delivering to the wound a delivery system, which may be a transdermal delivery system, the delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

In some embodiments, the rate of wound healing is more rapid when a compound disclosed herein is used. In some embodiments, the duration of healing is up to about 10%, 15%, 20%, 25%, 30%, 35%, 40%, 45%, 50%, 55%, 60%, 65%, 70%, 75%, or more than 75% shorter than if untreated. In some embodiments, with the use of a compound disclosed herein, the duration of wound healing is reduced by about 1, 2, 3, 4, 5, 6, 7, 8, 9, 10 days, or more than 10 days, than if untreated. Thus, in an exemplary embodiment, wound healing and/or regeneration of connective tissue at a wound site that would have a duration of 14 days without the administration, e.g., topical administration, of a serotonin receptor antagonist according to the present invention, e.g., an antagonist of a serotonin receptor 1B or an antagonist of the serotonin receptor 2B (or a combination thereof) is reduced by about 50%, to about 7 days; moreover, according to the presently described methods, the administration of a serotonin receptor antagonist decreases wound area size and/or promotes scarless healing of the skin wound compared to wound healing in the absence of the administration of an antagonist of the serotonin receptor 1B or an antagonist of the serotonin receptor 2B (or a combination thereof). In any of the foregoing embodiments, the compound is a selective antagonist of the serotonin receptor 1B or a selective antagonist of the serotonin receptor 2B.

In some embodiments, the degree of scarring of the healed wound when treated with a compound disclosed herein (a serotonin receptor antagonist) is less, e.g., scar-reduced, than that if the wound were untreated. In some embodiments, the scarring is reduced by up to about 10%, 15%, 20%, 25%, 30%, 35%, 40%, 45%, 50%, 55%, 60%, 65%, 70%, 75%, 80%, 85%, 90%, 95%, or is essentially scarless, by use of/administration of a compound disclosed herein, compared to no treatment. Thus, in some embodiments, the compounds and methods disclosed herein both increase the rate of wound healing and decrease the scarring of the healed wound. In an exemplary embodiment, the administration of a serotonin receptor antagonist according to the present invention, e.g., an antagonist of a serotonin receptor 1B or an antagonist of the serotonin receptor 2B (or a combination thereof) decreases wound scarring by 50%. In another example, both the healing time and the scarring are reduced by use of a compound disclosed herein. In another example, the healing time is reduced by about 50% and scar formation is reduced by more than 75%. In any of the foregoing embodiments, the compound is a selective antagonist of the serotonin receptor 1B or a selective antagonist of the serotonin receptor 2B.

Thus, in some embodiments, the compounds and methods disclosed herein increase the rate of wound healing and decrease the scarring of the healed wound. Thus, in some embodiments, the compounds and methods disclosed herein increase the rate of wound healing Thus, in some embodiments, the compounds and methods disclosed herein decrease the scarring of the healed wound.

Scar formation may be assessed by methods such as but not limited to those described by and/or in Oley et al., 2021, Post-skin incision scar tissue assessment using patient and observer scar assessment scales: A randomised controlled trial, Annals of Medicine and Surgery 71: 103006; Fearmonti et al., 2010, A review of scar scales and scar measuring devices, Eplasty Open Access Journal of Plastic Surgery 10:e43; and Nguyen et al., 2015, A review of scar assessment scales, Seminars in Cutaneous Medicine and Surgery 34: 28-36, each of which is incorporated herein by reference.

The following examples are presented in order to more fully illustrate exemplary embodiments of the invention. They should in no way be construed, however, as limiting the broad scope of the invention. Materials and methods may be found in PCT/US2019/059693, published as WO2020/096975; and/or in PCT/US2020/049529, published as WO2021/046428, each of which is incorporated herein by reference in its entirety.

### Example 1

### Suppression of Npas2 Regenerated Dermal Tissue of Mouse Splinted Skin Wound

The dermal tissue is composed of epithelial layer, connective tissue containing fibroblasts and collagen extracellular matrix (ECM) and sebaceous and sweat glands and hair follicles. The ideal wound healing requires acute stages of inflammation and angiogenesis followed by re-epithelialization and tissue remodeling. However, the tissue regeneration is difficult to achieve resulting in the granulation tissue and scarring.

Skin of experimental animals such as mice are similarly composed of multiple tissues; however excisional wound heals faster in part due to active wound contraction. Therefore, mouse wound healing model employs a silicone stent around the excisional skin, which prevents contraction allowing the healing through re-epithelialization and granulation tissue formation. The mouse splinted skin wound model has provided a common experimental platform, which allows investigation of critical molecules for skin wound healing using genetically manipulated mice. Knockout mutation of wound inflammation related molecules such as Sphingosine kinase-1 or mast cell proteases was reported to delay the wound closure and the proliferative phase of healing, respectively.

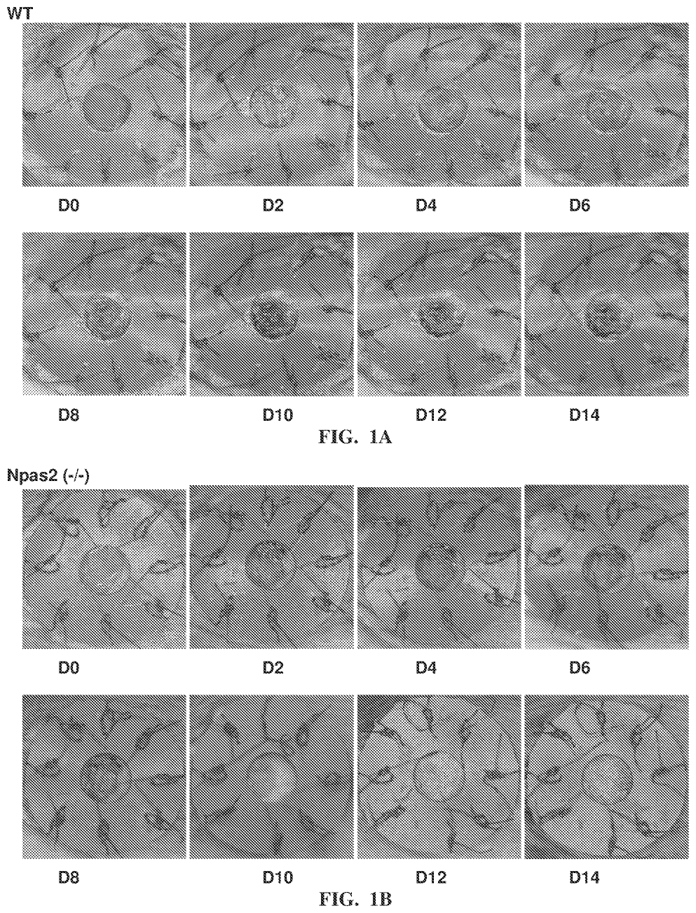

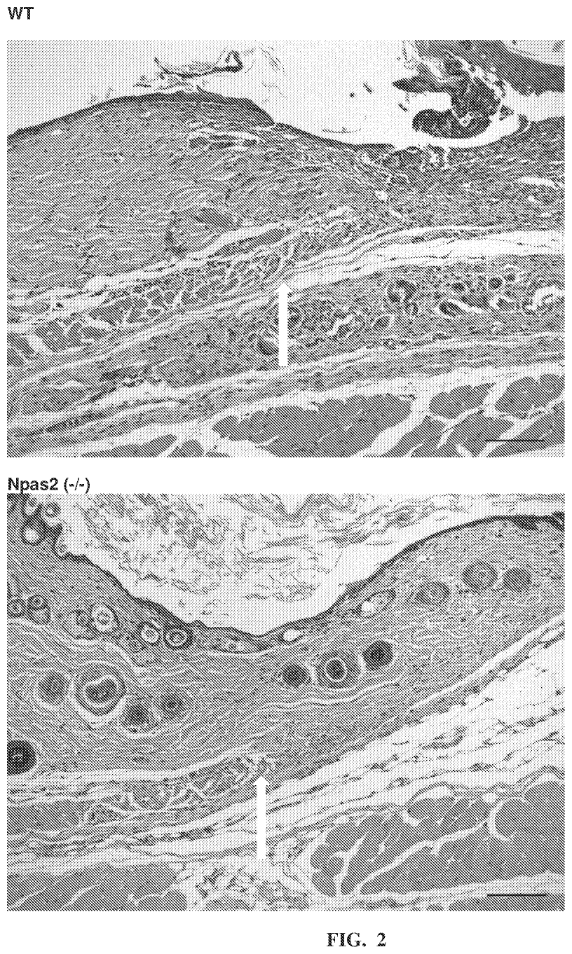

It has been reported that normal phenotype of dermal fibroblasts was maintained but myofibroblast differentiation was prevented in mice with knockout mutation of Npas2, a core circadian rhythm gene. Therefore, it is postulated that Npas2 KO mutation should accelerate skin wound healing. Results presented herein demonstrated that the splinted skin wound of Npas2 KO mice underwent accelerated wound healing (FIG. 1B) with a novel observation of skin tissue regeneration including sebaceous glands and hair follicles (FIG. 2).

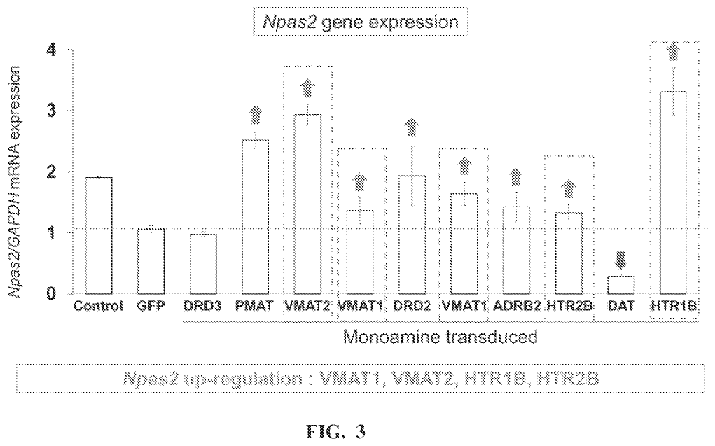

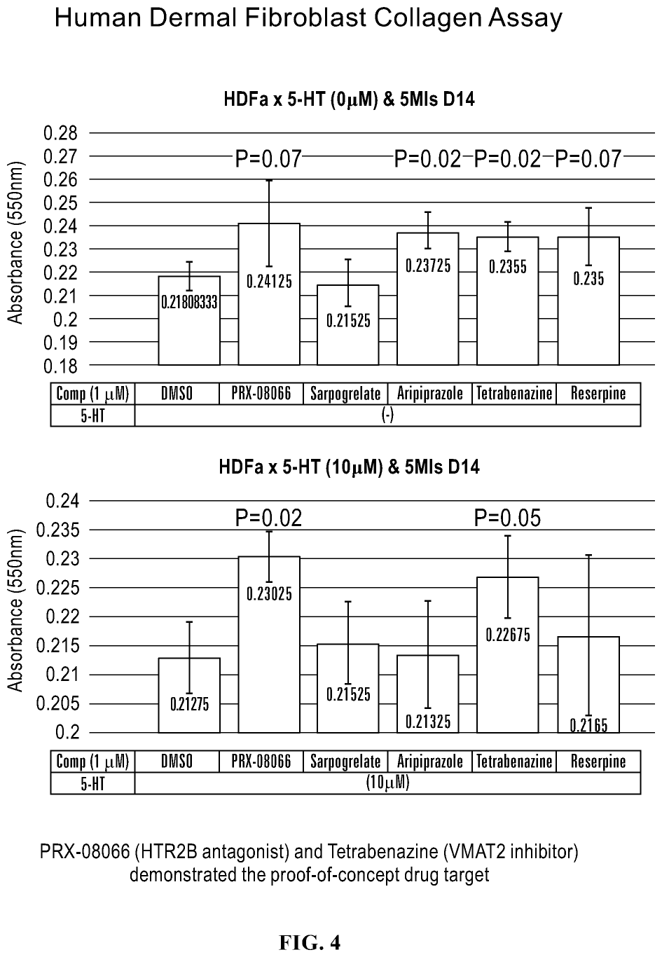

High throughput screening (HTS) of well-defined chemical compounds was performed using Npas2-LacZ reporter mouse skin fibroblasts. Chemical genomics analysis of hit compounds indicated the involvement of monoamine-related pathways for regulating Npas2 expression. To identify the regulatory mechanism of Npas2 in human dermal fibroblasts, a library of lentivirus expression vectors containing human monoamine-related genes was selected for over-expression study. As a result, over-expression of serotonin (5-HT) receptor 1B, 2B (HTR1B, 2B) upregulated Npas2 expression (FIG. 3). Results presented herein demonstrated that HTR1B, 2B antagonists decreased Npas2 expression and thus regenerate skin. A number of HTR1B, 2B antagonists were found to maintain human dermal fibroblast collagen synthesis phenotype in vitro (FIG. 4). It was further found that topical application of 5-HT antagonists (5 micromolar in 10% DMSO) accelerated mouse splinted skin wound healing in vivo (FIG. 5).

The therapeutic approaches for chronic skin wound are currently limited to address symptoms such as pain and infection. Advancing the wound edge to cover the ulcer lesion requires re-epithelialization and dermal connective tissue regeneration. Results presented herein describe the use of small molecule chemical compounds through topical application to chronic wound for accelerated and potentially regenerative wound healing.

### Example 2

Regeneration of Alveolar Bone and Periodontal Connective Tissues in the Mouse Ligature-Induced Periodontitis Model

Periodontal tissue is composed of alveolar bone and gingival/periodontal ligament collagen connective tissue. Therefore, periodontal therapies should aim to regenerate composite periodontal tissues of bone and gingival/periodontal ligament. Studies of tissue regeneration capacity of Npas2 KO mice have showed robust regeneration of experimental wound created in skin, calvarial bone and alveolar bone after tooth extraction. Therefore, the investigation was further extended for the composite tissue regeneration of periodontal tissues after ligature-induced periodontitis in mice. The placement of 5.0 silk suture around the maxillary second molar has been shown to induce periodontal inflammation and alveolar bone resorption.

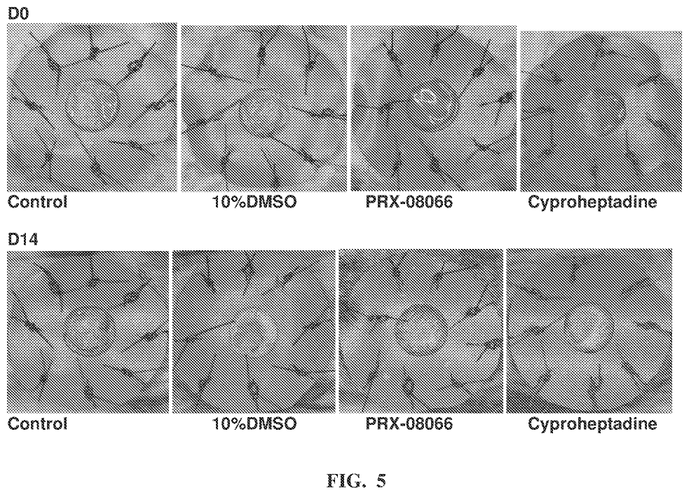

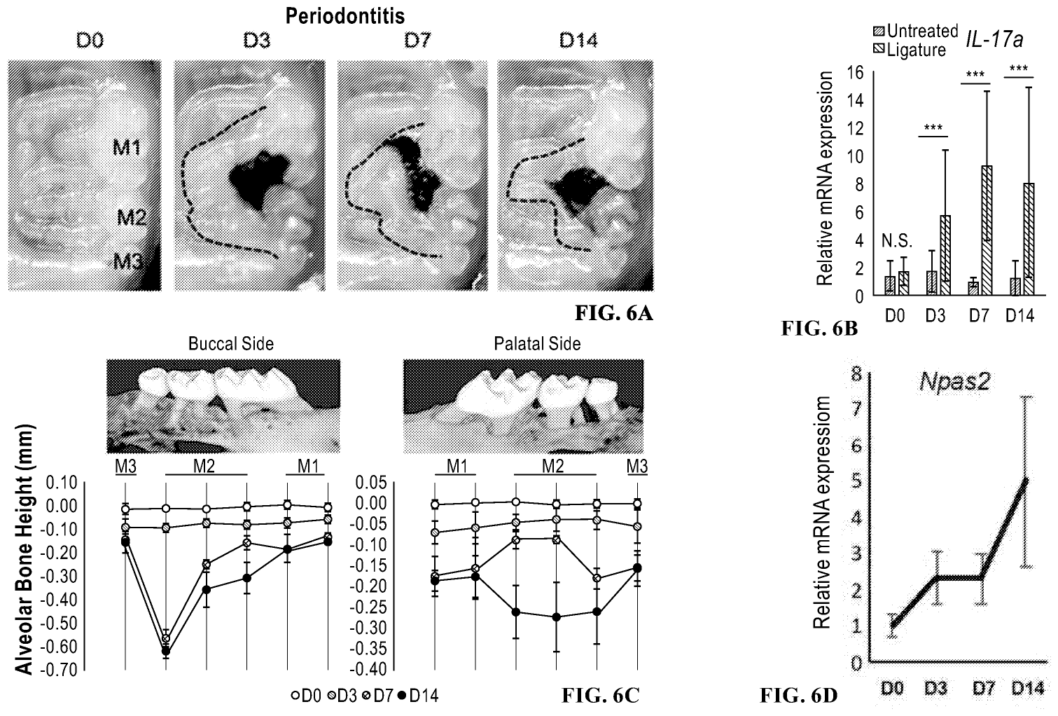

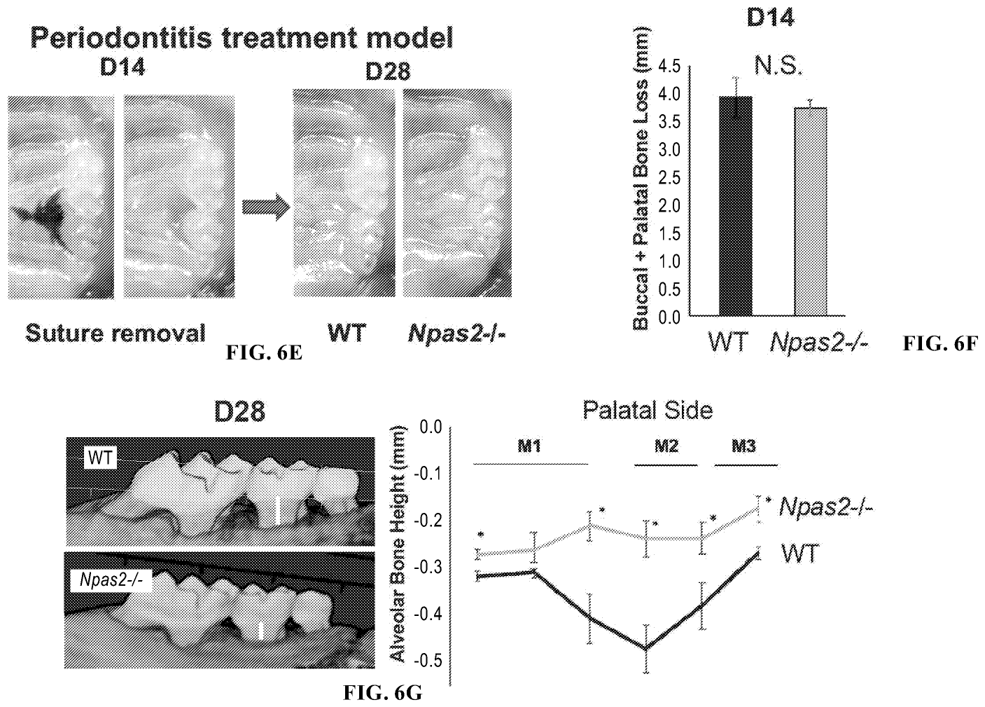

This commonly used experimental platform was duplicated in Npas2 KO mice. After 14 days, the silk suture ligature was removed, mimicking the dental professional cleaning (scaling and root plaining: SRP). This suture removal “SRP” treatment did not regenerate alveolar bone in wild type (WT) mice. However, after the suture removal treatment, Npas2 KO mice exhibited the regrowth of alveolar bone and regeneration of gingival/periodontal ligament collagen architecture (see FIGS. 6A-6G).

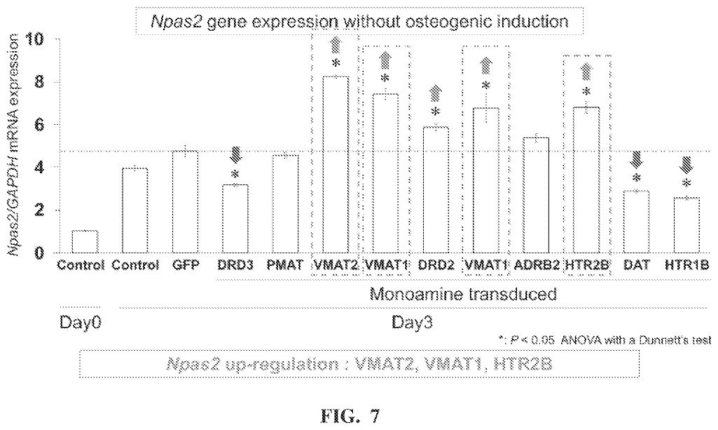

Mouse bone marrow mesenchymal stromal/stem cells (MSC) carrying Npas2-LacZ reporter system were used for HTS of small chemical compounds and hit compounds were identified by the capability of Npas2-LacZ expression modulation. These hit compounds were centered around the monoamine modulating compounds. To further identify the therapeutic targets, human MSC was transfected with lentivirus vectors expressing human monoamine-related genes. The outcome was not completely identical to that of the human dermal fibroblast experiment. However, HTR2B were commonly identified (FIG. 7).

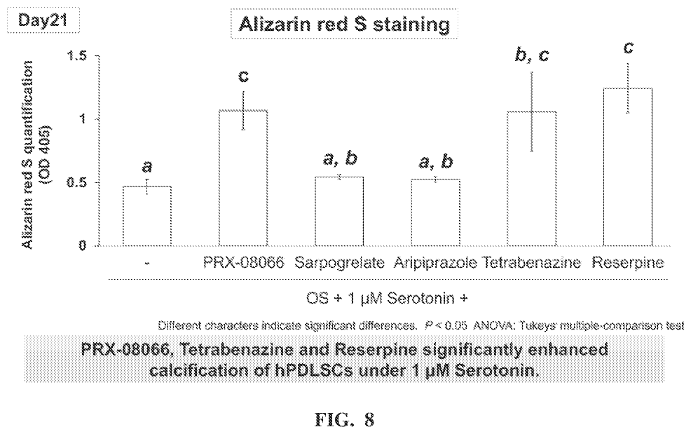

The chemical space specific compounds of HTR2B antagonists were tested for in vitro osteogenic differentiation using human periodontal ligament MSC (PDLSC), which further identified the serotonin receptor antagonist PRX-08066 as an effective compound (FIG. 8).

Treatment options that can predictably regenerate the lost periodontal tissue and alveolar bone remain a major clinical need. Therapeutic stimulation of bone regeneration resulted in clinical applications of recombinant growth factors. The biological rationale of growth factor therapies lies in the embryonic and developmental research, established that the bone morphogenetic protein (BMP) signaling pathway was important in bone formation. Because the adult tissue regeneration undergoes, at least in part, reiterated embryonic and developmental processes, the application of growth factors is believed to induce the signaling pathway necessary for the bone regeneration in periodontal defects. In the past decades, recombinant peptide therapeutics played an important role in medical/dental practices and over 60 peptide drugs are approved in the US and other major markets. Recombinant BMP-2 and PDGF are available for tooth extraction socket bone formation and periodontal tissue regeneration, respectively, for one-time surgical implantation to the defect site of alveolar bone.

Periodontitis is more prevalent in the geriatric population. Approximately 70% of this fastest growing segment of our society reported periodontal diseases with over 60% experiencing moderate to severe periodontitis. Currently, periodontitis of geriatric patients is primarily managed by supportive periodontal maintenance therapy: preventing progression or recurrence of periodontitis; and reducing or preventing tooth loss. The supportive maintenance therapy is composed of frequent professional cleaning such as periodontal debridement and SRP. Results presented herein envision to develop a topical therapy inducing periodontal tissue regeneration for geriatric patients, which can be combined with the supportive periodontal maintenance therapy without surgical approach. Small chemical topical application as discussed herein would be useful for long-term maintenance of periodontal tissue in the geriatric population.

It must be noted that companion animals such as dogs similarly suffer from extensive periodontitis. Some breeds of small dogs reported over 90% of periodontitis prevalence. The treatment options for companion dogs are limited to the professional cleaning and tooth extraction. The topical application of small chemical therapy discussed herein would also be effective in this veterinary patient segment.

### Example 3

### Dermal Fibroblasts Play a Central Role in Skin Wound Healing

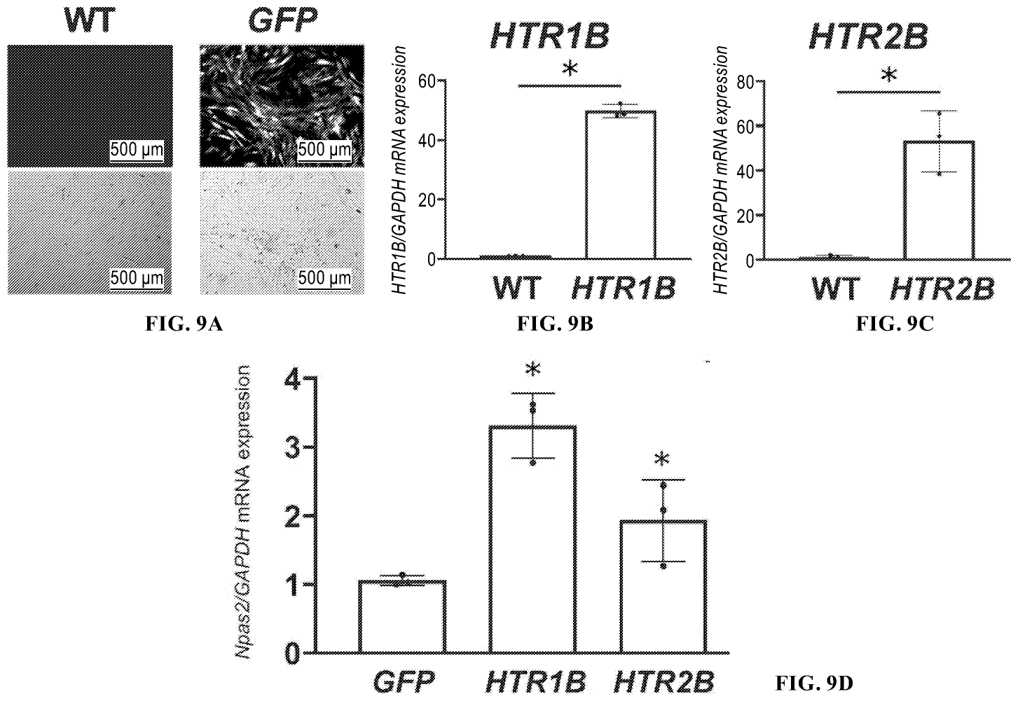

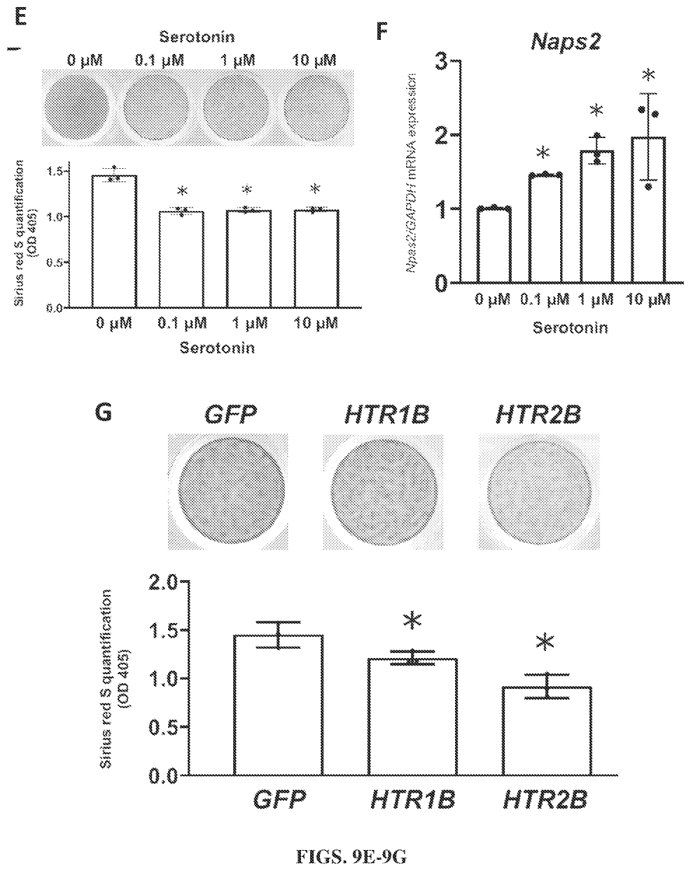

The dermis of granulation tissues during the skin wound healing is characterized as hypo-synthesis of dermal collagen, which then leads to the abnormal dermal thickening and the delayed development of hypertrophic scarring. This study transduced lentiviral vectors to human dermal fibroblasts and evaluated the effect of over-expression of HTR1B, HTR2B, VMAT2, DRD3 or DAT on Npas2 expression and dermal collagen synthesis. FIG. 9A shows using the lentivirus vector carrying GFP the successful lentivirus transduction to human dermal fibroblasts. FIGS. 9B and 9C show using quantitative PCR confirmed that the steady state mRNA level of HTR1B and HTR2B, respectively, were significantly increased after the lentiviral vectors were transduced.

The expression of Npas2 was significantly increased by HTR1B and HTR2B over-expression, supporting the potential role of serotonin (FIG. 9D). As described herein, wound-induced serotonin stimulates HTR1B and HTR2B and down-regulate dermal collagen synthesis.

Indeed, FIG. 9E shows serotonin supplementation decreased in vitro collagen synthesis of human dermal fibroblasts. And further, FIG. 9F shows that the serotonin-induced pathological behavior of dermal fibroblasts is mediated by the elevated expression of Npas2: serotonin supplementation increased Npas2 expression of human dermal fibroblasts.

Moreover, in the transduced cells, the pathological collagen hypo-synthesis of human dermal fibroblasts was mediated by HTR1B and HTR2B over-expression (FIG. 9G).

These data provide additional support for targeting HTR1B and HTR2B to mitigate the initial collagen hypo-synthesis. Specific antagonists of HTR1B and HTR2B as disclosed herein will reduce wound granulation tissue by regenerating dermal collagen connective tissue during the initial wound healing stages, ultimately preventing the formation of hypertrophic scarring, and resulting in a scarless, healed wound.

All patents, patent applications, and scientific publications cited herein are hereby incorporated by reference in their entirety.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A method for improving or accelerating wound healing in a subject in need thereof, comprising administering to a wound of the subject a therapeutically effective amount of a composition comprising an agent that suppresses expression of neuronal PAS domain protein 2 (Npas2), wherein the agent is a serotonin receptor antagonist.

2. The method of claim 1, wherein the serotonin receptor antagonist is an antagonist of the serotonin receptor 1B or serotonin receptor 2B.

3. The method of claim 2, wherein the antagonist of the serotonin receptor 1B is AR-A000002, Isamoltane (CGP-361A), Metergoline, Methiothepin, SB-216641, SB-236057 or Yohimbine.

4. The method of claim 2, wherein the antagonist of the serotonin receptor 2B is Agomelatine, Amisulpride, AM-1476, Aripiprazole, brilaroxazine hydrochloride, BF-1, BW 723C86, Cariprazine, Clozapine, Cyproheptadine, meta-Chlorophenyl-piperazine (mCPP), EGIS-7625, esamisulpride, F-16615, iferanserin, Ketanserin, LY-266097, LY-272,015, LY-287375, Metadoxine, piromelatine, Promethazine, PRX-08066, RS-127445 (MT 500), RS-127445, RQ-00310941, SB-204741, SB-206553, SB-200646A, SB-215505, SB-221284, SB 224289, SB-228357, SDZ SER-082, Tegaserod, Terguride, Ethyl 3-(4-Amino-1,3,5-triazaspiro[5.5]undeca-2,4-dien-2-ylamino)benzoate, 2-benzyl-5-hydroxy-4H-chromen-4-one, 5-hydroxy-2-(3-phenylpropyl)-4H-chromen-4-one, vabicaserin, XC-130, 4-(6-Isopropyl-1-methoxy-thioxanthen-9-yliden)-1-methyl-piperidine, 4-(6-Isopropyl-1-hydroxy-thioxanthen-9-yliden)-1-methyl-piperidine, 4-(6-Ethoxy-1-ethyl-thioxanthen-9-yliden)-1-methyl-piperidine, 4-(6-Ethoxy-1-methoxy-thioxanthen-9-yliden)-1-methyl-piperidine, 4-(6-Ethoxy-1hydroxy-thioxanthen-9-yliden)-1-methyl-piperidine, 4-(6-Methoxy-1-methoxy-thioxanthen-9-yliden)-1-methyl-piperidine, 4-(6-Dimethylamino-1-methoxy-thioxanthen-9-yliden)-1-methyl-piperidine, 4-(6-Dimethylamino-1-hydroxy-thioxanthen-9-yliden)-1-methyl-piperidine, 4-(3-Ethoxy-selenoxanthen-9-yliden)-1-methyl-piperidine, 4-(3-Ethoxy-1-methoxy-selenoxanthen-9-yliden)-1-methyl-piperidine, 4-(3-Ethoxy-1-hydroxy-selenoxanthen-9-yliden)-1-methyl-piperidine, 3-Ethoxy-9-(1-methyl-piperidine-4-ylidene)-9,10-dihydro-acridine, 6-Ethoxy-1-methoxy-9-(1-methyl-piperidine-4-ylidene)-9,10-dihydro-acridine, 6-Ethoxy-1-hydroxy-9-(1-methyl-piperidine-4-ylidene)-9, 10-dihydro-acridine, 4-(3-Ethoxy-5-ethyl-thioxanthen-9-yliden)-1-methyl-piperidine, 4-(3-Ethoxy-4-methyl-thioxanthen-9-yliden)-1-methyl-piperidine, 4-(3-Ethoxy-4-ethyl-thioxanthen-9-yliden)-1-methyl-piperidine, 4-(3-Ethoxy-thioxanthen-9-yliden)-1,3-dimethyl-piperidine, 4-(3,4-(Cyclopent-3′-oxy-1′-eno)-thioxanthen-9-yliden)-1-methyl-piperidine, 4-(3,4-(Cyclopent-4′-oxy-1′-eno)-thioxanthen-9-yliden)-1-methyl-piperidine, 4-(3,4-(Cyclopent-5‘-oxy-1’-eno)-thioxanthen-9-yliden)-1-methyl-piperidine or a dual 5-HT2a/5-HT2b antagonist selected from the group consisting of GSK-1606260, GSK-1645888, GSK-1742942, GSK-2054757, GSK-2080717 and SB-742888.

5. The method of claim 1, wherein the wound is a dermal wound.

6. The method of claim 1, wherein improved or accelerated wound healing comprises formation of a scar-reduced or scarless dermal wound.

7. The method of claim 5, wherein treating the dermal wound by administering the agent regenerates skin tissue that comprises sebaceous glands and hair follicles.

8. The method of claim 5, wherein the dermal wound comprises a periodontal wound.

9. The method of claim 8, wherein the periodontal wound comprises gingival connective tissue degeneration or alveolar bone resorption.

10. The method of claim 1, wherein the composition is administered by topical administration, transdermal administration, or subcutaneous administration.

11. The method of claim 1, wherein administration of the composition comprises delivering to the wound a delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

12. A method for regenerating connective tissue at a wound site in a subject in need thereof, comprising administering to a wound of the subject a therapeutically effective amount of a composition comprising an agent that suppresses expression of neuronal PAS domain protein 2 (Npas2), wherein the agent is a serotonin receptor antagonist.

13. The method of claim 12, wherein the serotonin receptor antagonist is an antagonist of the serotonin receptor 1B or serotonin receptor 2B.

14. The method of claim 13, wherein the antagonist of the serotonin receptor 1B is Aripiprazole, AR-A000002, Isamoltane (CGP-361A), Metergoline, Methiothepin, SB-216641, SB-236057 or Yohimbine.

15. The method of claim 13, wherein the antagonist of the serotonin receptor 2B is Agomelatine, Amisulpride, AM-1476, Aripiprazole, brilaroxazine hydrochloride, BF-1, BW 723C86, Cariprazine, Clozapine, Cyproheptadine, meta-Chlorophenyl-piperazine (mCPP), EGIS-7625, esamisulpride, F-16615, iferanserin, Ketanserin, LY-266097, LY-272,015, LY-287375, Metadoxine, piromelatine, Promethazine, PRX-08066, RS-127445 (MT 500), RS-127445, RQ-00310941, SB-204741, SB-206553, SB-200646A, SB-215505, SB-221284, SB 224289, SB-228357, SDZ SER-082, Tegaserod, Terguride, Ethyl 3-(4-Amino-1,3,5-triazaspiro[5.5]undeca-2,4-dien-2-ylamino)benzoate, 2-benzyl-5-hydroxy-4H-chromen-4-one, 5-hydroxy-2-(3-phenylpropyl)-4H-chromen-4-one, vabicaserin, XC-130, 4-(6-Isopropyl-1-methoxy-thioxanthen-9-yliden)-1-methyl-piperidine, 4-(6-Isopropyl-1-hydroxy-thioxanthen-9-yliden)-1-methyl-piperidine, 4-(6-Ethoxy-1-ethyl-thioxanthen-9-yliden)-1-methyl-piperidine, 4-(6-Ethoxy-1-methoxy-thioxanthen-9-yliden)-1-methyl-piperidine, 4-(6-Ethoxy-1hydroxy-thioxanthen-9-yliden)-1-methyl-piperidine, 4-(6-Methoxy-1-methoxy-thioxanthen-9-yliden)-1-methyl-piperidine, 4-(6-Dimethylamino-1-methoxy-thioxanthen-9-yliden)-1-methyl-piperidine, 4-(6-Dimethylamino-1-hydroxy-thioxanthen-9-yliden)-1-methyl-piperidine, 4-(3-Ethoxy-selenoxanthen-9-yliden)-1-methyl-piperidine, 4-(3-Ethoxy-1-methoxy-selenoxanthen-9-yliden)-1-methyl-piperidine, 4-(3-Ethoxy-1-hydroxy-selenoxanthen-9-yliden)-1-methyl-piperidine, 3-Ethoxy-9-(1-methyl-piperidine-4-ylidene)-9,10-dihydro-acridine, 6-Ethoxy-1-methoxy-9-(1-methyl-piperidine-4-ylidene)-9,10-dihydro-acridine, 6-Ethoxy-1-hydroxy-9-(I-methyl-piperidine-4-ylidene)-9, 10-dihydro-acridine, 4-(3-Ethoxy-5-ethyl-thioxanthen-9-yliden)-1-methyl-piperidine, 4-(3-Ethoxy-4-methyl-thioxanthen-9-yliden)-1-methyl-piperidine, 4-(3-Ethoxy-4-ethyl-thioxanthen-9-yliden)-1-methyl-piperidine, 4-(3-Ethoxy-thioxanthen-9-yliden)-1,3-dimethyl-piperidine, 4-(3,4-(Cyclopent-3‘-oxy-1’-eno)-thioxanthen-9-yliden)-1-methyl-piperidine, 4-(3,4-(Cyclopent-4‘-oxy-’-eno)-thioxanthen-9-yliden)-1-methyl-piperidine, 4-(3,4-(Cyclopent-5′-oxy-1′-eno)-thioxanthen-9-yliden)-1-methyl-piperidine or a dual 5-HT2a/5-HT2b antagonist selected from the group consisting of GSK-1606260, GSK-1645888, GSK-1742942, GSK-2054757, GSK-2080717 and SB-742888.

16. The method of claim 12, wherein the wound is a dermal wound.

17. The method of claim 12, wherein regenerating connective tissue at a wound site comprises formation of a scar-reduced or scarless dermal wound.

18. The method of claim 16, wherein treating the dermal wound by administering the agent regenerates skin tissue that comprises sebaceous glands and hair follicles.

19. The method of claim 16, wherein the dermal wound comprises a periodontal wound.

20. The method of claim 19, wherein the periodontal wound comprises gingival connective tissue degeneration or alveolar bone resorption.

21. The method of claim 19, wherein treating the periodontal wound with the agent results in regeneration of composite periodontal tissues.

22. The method of claim 21, wherein regeneration of composite periodontal tissues comprises one or more of regrowth of alveolar bone, regeneration of gingival ligament, and regeneration of periodontal ligament.

23. The method of claim 12, wherein the composition is administered by topical administration, transdermal administration, or subcutaneous administration.

24. The method of claim 12, wherein administration of the composition comprises delivering to the wound a delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

25. A method for reducing or preventing scar formation at a wound site during wound healing in a subject in need thereof, the method comprising administering to a wound of the subject a therapeutically effective amount of a composition comprising an agent that suppresses expression of neuronal PAS domain protein 2 (Npas2), wherein the agent is a serotonin receptor antagonist.

26. The method of claim 25, wherein the serotonin receptor antagonist is an antagonist of the serotonin receptor 1B or serotonin receptor 2B.

27. The method of claim 26, wherein the antagonist of the serotonin receptor 1B is Aripiprazole, AR-A000002, Isamoltane (CGP-361A), Metergoline, Methiothepin, SB-216641, SB-236057 or Yohimbine.

28. The method of claim 26, wherein the antagonist of the serotonin receptor 2B is Agomelatine, Amisulpride, AM-1476, Aripiprazole, brilaroxazine hydrochloride, BF-1, BW 723C86, Cariprazine, Clozapine, Cyproheptadine, meta-Chlorophenyl-piperazine (mCPP), EGIS-7625, esamisulpride, F-16615, iferanserin, Ketanserin, LY-266097, LY-272,015, LY-287375, Metadoxine, piromelatine, Promethazine, PRX-08066, RS-127445 (MT 500), RS-127445, RQ-00310941, SB-204741, SB-206553, SB-200646A, SB-215505, SB-221284, SB 224289, SB-228357, SDZ SER-082, Tegaserod, Terguride, Ethyl 3-(4-Amino-1,3,5-triazaspiro[5.5]undeca-2,4-dien-2-ylamino)benzoate, 2-benzyl-5-hydroxy-4H-chromen-4-one, 5-hydroxy-2-(3-phenylpropyl)-4H-chromen-4-one, vabicaserin, XC-130, 4-(6-Isopropyl-1-methoxy-thioxanthen-9-yliden)-1-methyl-piperidine, 4-(6-Isopropyl-1-hydroxy-thioxanthen-9-yliden)-1-methyl-piperidine, 4-(6-Ethoxy-1-ethyl-thioxanthen-9-yliden)-1-methyl-piperidine, 4-(6-Ethoxy-1-methoxy-thioxanthen-9-yliden)-1-methyl-piperidine, 4-(6-Ethoxy-1hydroxy-thioxanthen-9-yliden)-1-methyl-piperidine, 4-(6-Methoxy-1-methoxy-thioxanthen-9-yliden)-1-methyl-piperidine, 4-(6-Dimethylamino-1-methoxy-thioxanthen-9-yliden)-1-methyl-piperidine, 4-(6-Dimethylamino-1-hydroxy-thioxanthen-9-yliden)-1-methyl-piperidine, 4-(3-Ethoxy-selenoxanthen-9-yliden)-1-methyl-piperidine, 4-(3-Ethoxy-1-methoxy-selenoxanthen-9-yliden)-1-methyl-piperidine, 4-(3-Ethoxy-1-hydroxy-selenoxanthen-9-yliden)-1-methyl-piperidine, 3-Ethoxy-9-(1-methyl-piperidine-4-ylidene)-9,10-dihydro-acridine, 6-Ethoxy-1-methoxy-9-(1-methyl-piperidine-4-ylidene)-9,10-dihydro-acridine, 6-Ethoxy-1-hydroxy-9-(I-methyl-piperidine-4-ylidene)-9, 10-dihydro-acridine, 4-(3-Ethoxy-5-ethyl-thioxanthen-9-yliden)-1-methyl-piperidine, 4-(3-Ethoxy-4-methyl-thioxanthen-9-yliden)-1-methyl-piperidine, 4-(3-Ethoxy-4-ethyl-thioxanthen-9-yliden)-1-methyl-piperidine, 4-(3-Ethoxy-thioxanthen-9-yliden)-1,3-dimethyl-piperidine, 4-(3,4-(Cyclopent-3‘-oxy-1’-eno)-thioxanthen-9-yliden)-1-methyl-piperidine, 4-(3,4-(Cyclopent-4‘-oxy-’-eno)-thioxanthen-9-yliden)-1-methyl-piperidine, 4-(3,4-(Cyclopent-5′-oxy-1′-eno)-thioxanthen-9-yliden)-1-methyl-piperidine or a dual 5-HT2a/5-HT2b antagonist selected from the group consisting of GSK-1606260, GSK-1645888, GSK-1742942, GSK-2054757, GSK-2080717 and SB-742888.

29. The method of claim 25, wherein the wound is a dermal wound.

30. The method of claim 29, wherein treating the dermal wound by administering the agent comprises formation of a scar-reduced or scarless dermal wound.

31. The method of claim 29, wherein treating the dermal wound by administering the agent regenerates skin tissue that comprises sebaceous glands and hair follicles.

32. The method of claim 29, wherein the dermal wound comprises a periodontal wound.

33. The method of claim 32, wherein the periodontal wound comprises gingival connective tissue degeneration or alveolar bone resorption.

34. The method of claim 25, wherein the composition is administered by topical administration, transdermal administration, or subcutaneous administration.

35. The method of claim 25, wherein administration of the composition comprises delivering to the wound a delivery system comprising deformable nanoscale vesicles encapsulating the agent, a microneedle coated with the agent, a solid polymer matrix having the agent incorporated therein, a transdermal patch comprising the agent, a transdermal gel comprising the agent, or a transdermal spray comprising the agent.

36. The method of claim 25 wherein the wound exhibits improved or accelerated wound healing.
